(19) United States
(12) Patent Application Publication
(10) Pub. No.: US 2024/0367346 A1
(43) Pub. Date: Nov. 7, 2024

(54) METHODS AND SYSTEMS FOR HANDLING CONCRETE MIXER TRUCK HAVING RETURN CONCRETE

(71) Applicant: COMMAND ALKON INCORPORATED, Birmingham, AL (US)

(72) Inventor: Rob PIOSIK, Heerlen (NL)

(73) Assignee: COMMAND ALKON INCORPORATED, Birmingham, AL (US)

(21) Appl. No.: 18/565,821

(22) PCT Filed: Apr. 20, 2022
(86) PCT No.: PCT/US2022/025567

§ 371 Date: Nov. 30, 2023

Related U.S. Application Data
(60) Provisional application No. 63/197,617, filed on Jun. 7, 2021.

(51) Int. Cl. B28C 5/42; B28C 7/02; G01N 33/38

(52) U.S. Cl. CPC B28C 5/422; B28C 7/024; G01N 33/383

(57) ABSTRACT

There is described a computer-implemented method for handling a mixer truck containing a return concrete load. The method generally has: accessing return concrete data including at least quantity data indicative of a quantity of the return concrete load contained in the drum of the mixer truck and composition data indicative of a composition of the return concrete load contained in the drum; accessing ticket data including job tickets each including a ticket specification; establishing a list of eligible job tickets by comparing the return concrete data to each job ticket, and including a given one of the job tickets in the list contingent upon finding a match between the ticket specification and the return concrete data; and generating a signal indicative of the established list of eligible job tickets.

### BACKGROUND

Fresh concrete is formed of a mixture of ingredients typically including cement, aggregate and water in given proportions, but there are various recipes and many of these include additional ingredients. The ingredients are typically transported inside a rotary drum of a concrete mixer truck where the fresh concrete mixture can be mixed and then continuously agitated prior to being unloaded at a job site. Fresh concrete is a perishable rheological substance, its slump and temperature typically evolve over time and this evolution can eventually make the fresh concrete unusable for its intended purpose, or require introduction of additives or water. Agitation by the rotation of the drum and internal paddles is very significant in preserving the freshness of the concrete during transport, but only to a certain extent.

Around many cities, it is typical to have a number of job sites in simultaneous operation, overall requiring high volumes of fresh concrete of same or differing requirements, and the operations involving fresh concrete can be relatively complex. For instance, different job sites can be associated to a same, or different buyers (customers). There can be a number of batch plants, associated to a same or different producers, available for fulfilling customer requests and filling mixer trucks with suitable fresh concrete. An example of such a mixer truck is shown in FIG. 1. Different batch plants can offer the same, or different recipes of concrete. To complicate things even further, the mixer trucks may be managed by yet a third party, commonly referred to as a hauler, though in some cases the mixer trucks can be the property of the buyer or of the producer. In any given day, many different orders can be transacted in parallel, giving rise to several communications between buyers, producers and trucks, such as schematized in FIG. 2.

A typical order is placed by a buyer. Typically, though not always, the buyer is more concerned about requirements of the concrete, or specifications, than the details of the exact recipe. The order can thus be based on specifications which may make abstraction of the exact recipe. Such specifications typically set a minimum strength for the concrete, and a required volume, but depending on the circumstances, the specifications can set a number of additional parameters such as aggregate size, water content, or other specific conditions. Orders also specify a required quantity of concrete.

To satisfy the order, a match must be established between the specifications and one or more of the recipes which are made available by at least one batch plant located at a suitable distance from an available truck and from a corresponding job site. If this match is established, the transaction can proceed, and one or more concrete trucks can be directed to one or more batch plants to receive a corresponding fresh concrete load, and then to the job site to deliver the fresh concrete load.

In high volume operations, to satisfy economic and logistics considerations, such operations can be performed in the framework of a coordination scheme which can include protocols designed to facilitate the various communications between the buyer(s), the batch plant(s), and the truck(s) or haulers. In particular, in many jurisdictions, the liability rests on the producer(s)/batch plant(s) in terms of ensuring that the fresh concrete load which is ultimately unloaded at a given job site satisfies the buyer's specifications, and several, partially redundant, safety measures can be in place to avoid any significant likelihood of mistakes.

While the existing technologies associated to fresh concrete operations were satisfactory to a certain degree, there always remains room for improvement. In particular, it is relatively common for some of the initially loaded fresh concrete to remain in the truck subsequently to unloading at the job site. This can stem from the fact that the producer or buyer wanted to make sure that the required volume of concrete was met, erring on the side of caution and requesting or delivering more than necessary, or simply due to a difference between the quantity ordered. Some jurisdictions, such as the United States for instance, allow the re-use of return concrete within certain conditions, which are set out in regulations such as national or regional standards. In the United States, an existing national standard at the time of filing this specification was under ASTM. Such conditions can be relatively complex and require the re-use to occur within a predefined, limited quantity of time, impose temperature limits on the fresh concrete, and/or impose specific limits to the quantity of water added into the concrete subsequently to the initial mixing to name a few examples. Determining whether regulations are satisfied can require using information held by more than one of the parties involved in the transaction. Moreover, it can be required to monitor whether the standard continues to be satisfied over time. Likely at least in part due to the complexities imposed by such conditions and/or the overall complexities of the logistics surrounding the handling of fresh concrete, available return concrete often ends up being dumped, which often incurs a penalty in the form of a dumping cost, or otherwise not recovering much value. This can occur quite frequently even in scenarios where i) its re-use is permissible in the jurisdiction in question, ii) a job site would have agreed to pay a suitable fee to purchase the return concrete, and iii) the return concrete could, at least theoretically, have been delivered to that job site within a time frame satisfying existing conditions for the re-use.

There thus remains a significant need for technological improvements which would allow to retrieve a greater value from return concrete, or otherwise facilitate the re-use of return concrete in a manner to satisfy existing standards or regulations in addition to satisfying customer and efficiency requirements.

### SUMMARY

Coordination schemes designed to facilitate communications between buyer(s), producer(s) (batch plants) and truck(s) can involve truck sensors and computers configured to communicate with one another over telecommunications network. Coordination schemes can further include a coordinator platform configured to act as an intermediary to some or all of the communications between the parties involved. A coordinator platform can include a plurality of software layers, an example of which is presented in FIG. 3. Such a coordinator platform can include a plurality of software applications running or otherwise providing input and output functionalities on different computers associated to the parties involved.

For instance, the buyer can order concrete from the producer by phone call, fax, internet, etc., or have one or more computers which enable the buyer to make orders electronically in the form of data which can be referred to as job tickets. The job tickets can include the various specifications associated to the order and be communicated via the telecommunications network. The software used to perform the latter functions can be referred to as the buyer layer.

The producer can have one or more computers which enable to input its available recipes electronically in the form of data which can be referred to as offer tickets. The offer tickets can include a plurality of specifications associated to available recipes. The offer tickets can be communicated via the telecommunications network, for example. The software used to perform the latter functions can be referred to as the producer layer.

A coordinator layer, which can alternately be referred to as a dispatch layer, can further be provided. The coordinator layer can have one or more computers which have software configured to facilitate the establishment of matches between job tickets and order tickets. The coordinator layer software can be designed to display a list of job tickets and a list of offer tickets on a display screen made available to a human user which can be referred to as a dispatcher or coordinator for instance. In the highly technological era in which we live in, various configurations are possible for the implementation of the coordinator layer. In one example, the coordinator layer can be in the form of computer program product stored on computers owned by the batch plants, or perhaps alternately computers owned by the buyers, for instance. In still another example, the coordinator layer can be handled by a third party and can run in the cloud-based platform, such as on server computers remote to both the buyer and producer computers (e.g., Amazon web services), and communicatively coupled therewith via the telecommunications network. The dispatcher or coordinator can be an employee of such a third party, or be an employee of the buyer or producer, for instance, to whom the relevant options are displayed in a manner to request user input as to the choice of the dispatcher or coordinator.

In accordance with a first aspect of the present disclosure, there is provided a computer-implemented method for handling a mixer truck containing a return concrete load, the method comprising: accessing return concrete data including at least quantity data indicative of a quantity of the return concrete load contained in the drum of the mixer truck and composition data indicative of a composition of the return concrete load contained in the drum; accessing ticket data including a plurality of job tickets each including a ticket specification; establishing a list of eligible job tickets by comparing the return concrete data to each job ticket, and including a given one of the job tickets in the list contingent upon finding a match between the ticket specification and the return concrete data; and generating a signal indicative of the established list of eligible job tickets.

Further in accordance with the first aspect of the present disclosure, the method can for example further comprise accessing regulation data including a regulation specification, and performing said establishing contingent upon finding a match between the regulation specification and the return concrete data of the return concrete load.

Still further in accordance with the first aspect of the present disclosure, the ticket specification can for example include at least one of a minimum quantity requirement and a composition requirement.

Still further in accordance with the first aspect of the present disclosure, said return concrete data can for example further include concrete parameter data indicative of at least another parameter of the return concrete contained in the drum of the mixer truck, each job ticket further including a requirement for the other parameter, the list of eligible job tickets including the given one of the job tickets in the list contingent upon finding an additional match between the requirement for the other parameter and the parameter data of the return concrete data.

Still further in accordance with the first aspect of the present disclosure, the other parameter can for example be at least one of water content, temperature, concrete strength, aggregate size, and slump.

Still further in accordance with the first aspect of the present disclosure, the method can for example further comprise at least one of displaying the established list of use eligible job tickets on a graphical user interface, communicating the established list of eligible job tickets over an accessible network and storing the established list of eligible job tickets on an accessible memory.

Still further in accordance with the first aspect of the present disclosure, said accessing return concrete data can for example further include at least one of receiving the quantity data from a controller associated to the mixer truck, receiving the composition data from the mixer truck and receiving the composition data from the producer.

Still further in accordance with the first aspect of the present disclosure, upon selecting a given one of the eligible job ticket, the method can for example include removing the selected one of the eligible job tickets from the list.

Still further in accordance with the first aspect of the present disclosure, the return concrete data can for example further include location data indicative of a location of the return concrete contained in the drum of the mixer truck, each job ticket further including a jobsite location, the list of eligible job tickets including the given one of the job tickets in the list further contingent upon determining that a match between the jobsite location and the location data of the return concrete data.

Still further in accordance with the first aspect of the present disclosure, the list of eligible job tickets can for example further include the given one of the job ticket contingent upon finding that a travel time from the location of the mixer truck to the location of the job site is below given travel time threshold.

In accordance with a second aspect of the present disclosure, there is provided a system for handling a mixer truck containing a return concrete load, the system comprising: a coordinator device communicatively coupled to an external network, the coordinator device having a processor and a memory having stored thereon instructions that when executed by the processor perform the steps of: accessing return concrete data including at least quantity data indicative of a quantity of the return concrete load contained in the drum of the mixer truck and composition data indicative of a composition of the return concrete load contained in the drum; accessing ticket data including a plurality of job tickets each including a ticket specification; establishing a list of eligible job tickets by comparing the return concrete data to each job ticket, and including a given one of the job tickets in the list contingent upon finding a match between the ticket specification and the return concrete data; and generating a signal indicative of the established list of eligible job tickets.

Further in accordance with a second aspect of the present disclosure, the system can for example further comprise accessing regulation data including a regulation specification, and performing said establishing contingent upon finding a match between the regulation specification and the return concrete data of the return concrete load.

Still further in accordance with a second aspect of the present disclosure, the system can for example further comprise the ticket specification includes at least one of a minimum quantity requirement and a composition requirement.

Still further in accordance with a second aspect of the present disclosure, said return concrete data can for example further include concrete parameter data indicative of at least another parameter of the return concrete contained in the drum of the mixer truck, each job ticket further including a requirement for the other parameter, the list of eligible job tickets including the given one of the job tickets in the list contingent upon finding an additional match between the requirement for the other parameter and the parameter data of the return concrete data.

Still further in accordance with a second aspect of the present disclosure, the other parameter can for example be at least one of water content, temperature, concrete strength, aggregate size, and slump.

Still further in accordance with a second aspect of the present disclosure, the system can for example further comprise at least one of displaying the established list of use eligible job tickets on a graphical user interface, communicating the established list of eligible job tickets over an accessible network and storing the established list of eligible job tickets on an accessible memory.

Still further in accordance with a second aspect of the present disclosure, said accessing return concrete data can for example include at least one of receiving the quantity data from a controller associated to the mixer truck, receiving the composition data from the mixer truck and receiving the composition data from the producer.

Still further in accordance with a second aspect of the present disclosure, upon selecting a given one of the eligible job ticket, the system can for example further include a step of removing the selected one of the eligible job tickets from the list.

Still further in accordance with a second aspect of the present disclosure, the return concrete data can for example further include location data indicative of a location of the return concrete contained in the drum of the mixer truck, each job ticket further including a jobsite location, the list of eligible job tickets including the given one of the job tickets in the list further contingent upon determining that a match between the jobsite location and the location data of the return concrete data.

Still further in accordance with a second aspect of the present disclosure, the list of eligible job tickets can for example include the given one of the job ticket contingent upon finding that a travel time from the location of the mixer truck to the location of the job site is below given travel time threshold.

In accordance with a third aspect of the present disclosure, there is provided a computer-implemented method for handling a plurality of mixer trucks each containing a return concrete load, the method comprising: accessing a job ticket including a ticket specification; accessing return concrete data for the plurality of mixer trucks, the return concrete data including at least quantity data indicative of a quantity of a return concrete load contained in a drum of a given one of the mixer trucks and composition data indicative of a composition of the return concrete load contained in the drum of the given one of the mixer trucks; establishing a list of eligible return concrete loads by comparing the job ticket to the return concrete data of each return concrete load, and including a given one of the return concrete loads in the list contingent upon finding a match between the ticket specification and the return concrete data; and generating a signal indicative of the established list of eligible return concrete loads.

Further in accordance with a third aspect of the present disclosure, the method can for example further comprise accessing regulation data including a regulation specification, and including the given one of the return concrete loads in the list contingent upon finding a match between the regulation specification and the return concrete data of the given one of the return concrete loads.

Still further in accordance with a third aspect of the present disclosure, the method can for example further comprise the ticket specification includes at least one of a minimum quantity requirement and a composition requirement.

Still further in accordance with a third aspect of the present disclosure, said return concrete data for each mixer truck can for example further include concrete parameter data indicative of at least another parameter of the return concrete contained in the drum of the corresponding mixer truck, the job ticket further including a requirement for the other parameter, the list of eligible return concrete loads including the given one of the return concrete loads in the list contingent upon finding an additional match between the requirement for the other parameter and the parameter data of the return concrete data.

Still further in accordance with a third aspect of the present disclosure, the other parameter can for example be at least one of water content, temperature, concrete strength, aggregate size, and slump.

Still further in accordance with a third aspect of the present disclosure, the method can for example further comprise at least one of displaying the established list of use eligible return concrete loads on a graphical user interface, communicating the established list of eligible return concrete loads over an accessible network and storing the established list of eligible return concrete loads on an accessible memory.

Still further in accordance with a third aspect of the present disclosure, said accessing return concrete data can for example include at least one of receiving the quantity data from a controller associated to the mixer truck, receiving the composition data from the mixer truck and receiving the composition data from the producer.

Still further in accordance with a third aspect of the present disclosure, upon selecting a given one of the eligible return concrete loads, the method can for example include removing the selected one of the eligible return concrete loads from the list.

Still further in accordance with a third aspect of the present disclosure, each return concrete data further can for example include location data indicative of a location of the return concrete contained in the drum of the mixer truck, the job ticket further including a jobsite location, the list of eligible return concrete loads including the given one of the return concrete loads in the list further contingent upon determining that a match between the jobsite location and the location data of the return concrete data.

Still further in accordance with a third aspect of the present disclosure, the list of eligible return concrete loads can for example include the given one of the return concrete loads contingent upon finding that a travel time from the location of the mixer truck to the location of the job site is below given travel time threshold.

In accordance with a fourth aspect of the present disclosure, there is provided a system for handling a plurality of mixer trucks each containing a return concrete load, the system comprising: a coordinator device communicatively coupled to an external network, the coordinator device having a processor and a memory having stored thereon instructions that when executed by the processor perform the steps of: accessing a job ticket including a ticket specification; accessing return concrete data for the plurality of mixer trucks, the return concrete data including at least quantity data indicative of a quantity of a return concrete load contained in a drum of a given one of the mixer trucks and composition data indicative of a composition of the return concrete load contained in the drum of the given one of the mixer trucks; establishing a list of eligible return concrete loads by comparing the job ticket to the return concrete data of each return concrete load, and including a given one of the return concrete loads in the list contingent upon finding a match between the ticket specification and the return concrete data; and generating a signal indicative of the established list of eligible return concrete loads

In accordance with a fifth aspect of the present disclosure, there is provided a computer-implemented method for handling mixer trucks containing return concrete loads, the method comprising: accessing return concrete data including a batching time and a temperature of the return concrete load for each one of a plurality of mixer trucks containing return concrete loads; accessing regulation data including a maximum temperature for return concrete re-use and a maximum elapsed time since batching; and generating a list of eligible return concrete loads, the list of eligible return concrete loads including each return concrete load which said return concrete data match said regulation data.

In accordance with a sixth aspect of the present disclosure, there is provided a system for handling mixer trucks containing return concrete loads, the system comprising: a coordinator device communicatively coupled to an external network, the coordinator device having a processor and a memory having stored thereon instructions that when executed by the processor perform the steps of: accessing return concrete data including a batching time and a temperature of the return concrete load for each one of a plurality of mixer trucks containing return concrete loads; accessing regulation data including a maximum temperature for return concrete re-use and a maximum elapsed time since batching; and generating a list of eligible return concrete loads, the list of eligible return concrete loads including each return concrete load which said return concrete data match said regulation data.

It will be understood that the expression “computer” as used herein is not to be interpreted in a limiting manner. It is rather used in a broad sense to generally refer to the combination of some form of one or more processing units and some form of memory system accessible by the processing unit(s). The memory system can be of the non-transitory type. The use of the expression “computer” in its singular form as used herein includes within its scope the combination of a two or more computers working collaboratively to perform a given function. Moreover, the expression “computer” as used herein includes within its scope the use of partial capabilities of a given processing unit.

It will be understood that a computer can perform functions or processes via hardware or a combination of both hardware and software. For example, hardware can include logic gates included as part of a silicon chip of a processor. Software (e.g., application, process) can be in the form of data such as computer-readable instructions stored in a non-transitory computer-readable memory accessible by one or more processing units. With respect to a computer or a processing unit, the expression “configured to” relates to the presence of hardware or a combination of hardware and software which is operable to perform the associated functions. A processor, controller, and/or memory can be local in some embodiments, or partially or entirely remote, distributed and/or virtual in other embodiments.

Many further features and combinations thereof concerning the present improvements will appear to those skilled in the art following a reading of the instant disclosure.

### DESCRIPTION OF THE FIGURES

In the figures,

FIG. 1 is a schematic view of an example of a concrete mixer truck;

FIG. 2 is a schematic view of an example interaction scheme between parties involved in the delivery of a fresh concrete load to a job site;

FIG. 3 is a schematic view of example software connectively layers and data flows which can be used to facilitate or implement some or all of the interactions between parties involved in the delivery of a fresh concrete load to a job site;

FIG. 4 is a block diagram showing an example embodiment of a computer;

FIG. 5 is a diagram showing a software layer communication scheme in accordance with one embodiment;

FIG. 6 is a flow chart representing a portion of an example process for addressing an occurrence of return concrete, in accordance with one or more embodiments;

FIG. 7 is a chart representing an example of parties and truck movements in the delivery of a fresh concrete load to a job site, and different return concrete uses;

FIG. 8 is a cross-sectional view of the concrete mixer truck of FIG. 1 taken along the corresponding cross-section lines of FIG. 1;

FIG. 9 is a flow chart representing a second portion of an example process for addressing an occurrence of return concrete, in accordance with one or more embodiments;

FIG. 10 is a flow chart showing an example process for generating a list of eligible job tickets for a given return concrete load, in accordance with one or more embodiments;

FIG. 11 is a block diagram of an example system configured to perform the process of FIG. 10, in accordance with one or more embodiments;

FIG. 12 is a block diagram showing a detailed implementation of the process of FIG. 10;

FIG. 13 is a flow chart of a detailed implementation of an example process of handling return concrete loads, in accordance with one or more embodiments;

FIG. 14 is a flow chart of an example process for generating a list of eligible return concrete loads for a given job ticket, in accordance with one or more embodiments;

FIG. 15 is a block diagram of an example system configured to perform the process of FIG. 14;

FIG. 16 is a flow chart of another example process for generating a list of eligible return concrete loads matching regulatory requirements, in accordance with one or more embodiments

FIG. 17 is a block diagram of an example system configured to perform the process of FIG. 16, outputting a list of eligible return concrete loads;

FIG. 18 is a block diagram of an example system configured to perform the process of FIG. 16, outputting a sub list of eligible job tickets; and

FIG. 19 is a block diagram showing a detailed implementation of the process of FIG. 16.

### DETAILED DESCRIPTION

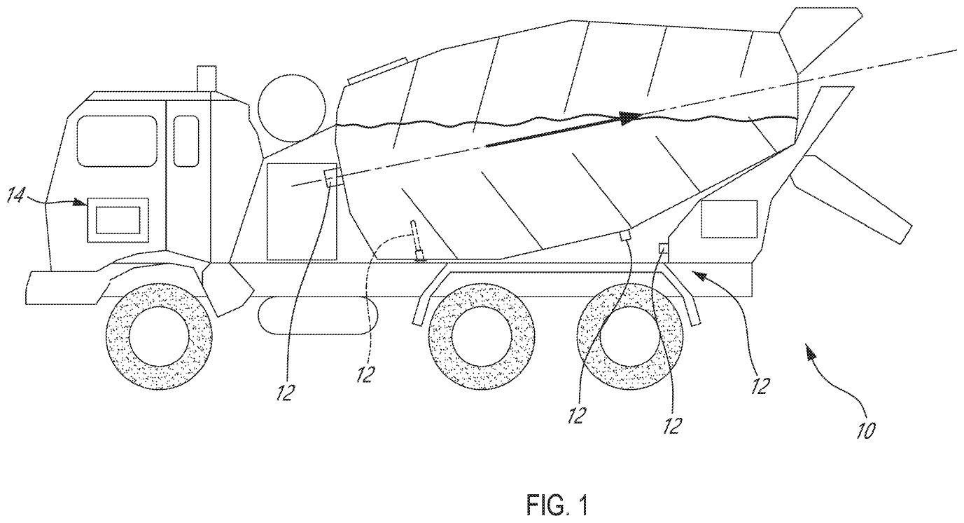

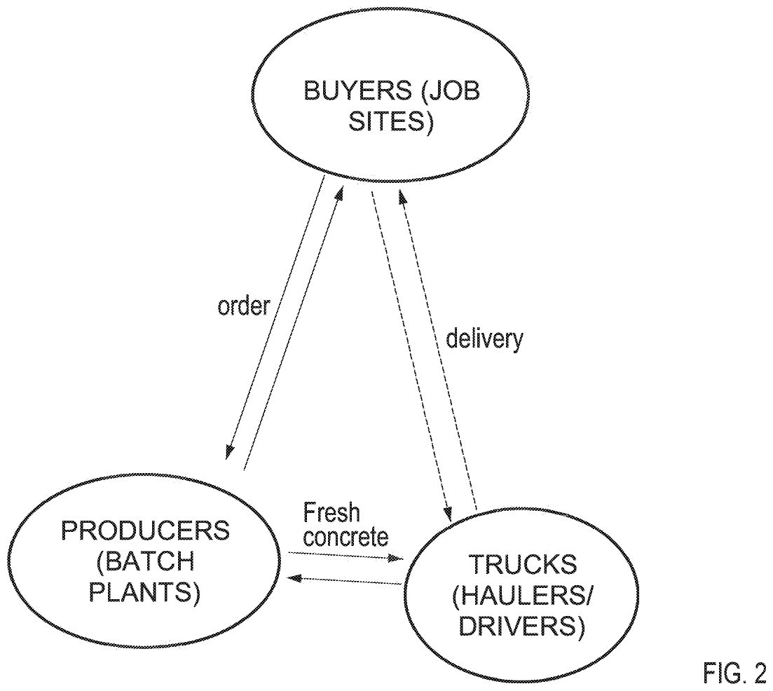

FIG. 1 shows an example of a concrete mixer truck 10 which can be equipped with one or more sensors 12 used to measure different measurands such as slump, viscosity, yield, temperature, quantity of fresh concrete (e.g., volume, weight), drum rotation speed, drum rotation orientation, occurrence of unloading, termination of unloading, water content, flow rate of added water or additive, location (e.g., GPS), density, air content, etc. Concrete mixer trucks 10 can be equipped or otherwise associated to an on-board computer 14 which can be communicatively coupled to the one or more sensors in a wired or wireless manner to receive signals indicative of the measurands being sensed. The computer 14 can have software configured to calculate or otherwise determine a value of some measurands based on raw signals from a sensor. Concrete mixer trucks 10 can be further equipped with a transmitter connected to the computer 14 and operable to allow the computer 14 to communicate with remote computers via a telecommunications network such as the Internet. The on-board computer 14 can be owned by an owner of the truck who may be somewhat independent from the truck driver, and/or be a computer, such as a tablet, smartphone, or smartwatch for instance, owned by the truck driver. Such a computer(s) can be enabled to present options or directions to the truck driver. Such a computer(s) can also be enabled to request confirmations or feedback from the truck driver and/or from devices or sensors associated to the truck. The software used to perform the various latter functions can be referred to as the truck layer.

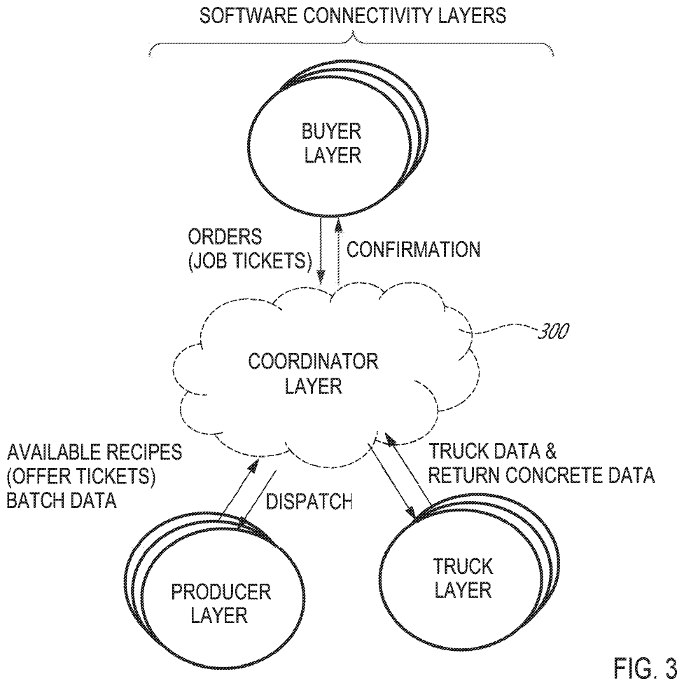

Referring to FIG. 3, once a job ticket has been matched to an offer ticket, the coordinator layer 300 can direct a concrete mixer truck to a batch plant to receive a load of fresh concrete corresponding to the job ticket and offer ticket match. The job ticket and offer ticket matches can be referred to as dispatch data. In practice, the fresh concrete loaded into the truck at the batch plant can slightly differ from what was theoretically agreed in the dispatch data, and perhaps the most common discrepancy is a slight difference between theoretical quantity of concrete agreed upon and actual quantity of fresh concrete loaded into the drum. It is common for the batch plant to provide detailed specifications, including exact quantity of concrete loaded into the drum, quantity of cement included in the concrete, time at which the batch was loaded, etc. following the loading operation. These detailed specifications can be referred to as batch data and can be communicated to the buyer, to the truck, and/or to the coordinator layer 300 for instance. The batch data can serve as a reference as to what was loaded into the truck at the time of loading, but in many cases, it can be needed to rely on the truck's system for providing information about what may have happened to the concrete subsequently to the loading operation, such as in terms of water and/or additive addition, or the time or place at which certain events take place.

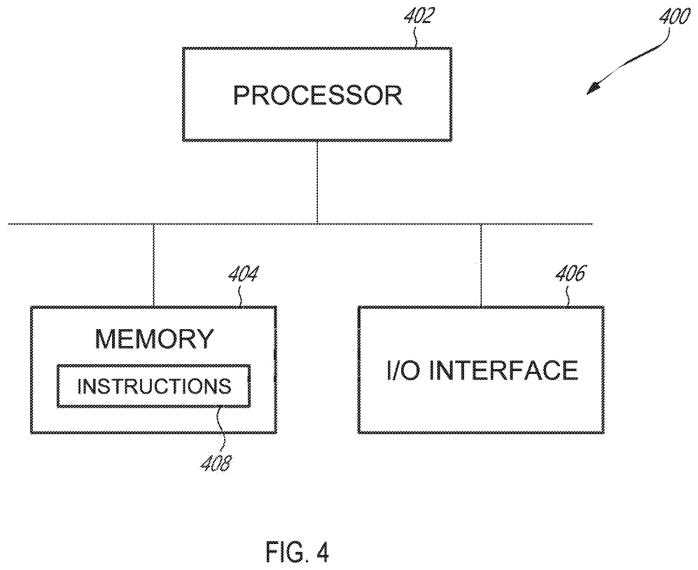

The on-board computer 14 can be provided as a combination of hardware and software components. The hardware components can be implemented in the form of a computer 400, an example of which is described with reference to FIG. 4. Moreover, the software components of the on-board computer 14 can be implemented in the form of one or more software applications.

Referring to FIG. 4, the computer 400 can have a processor 402, a memory 404, and I/O interface 406. Instructions 408 for running the one or more software applications can be stored on the memory 404 and accessible by the processor 402.

The processor 402 can be, for example, a general-purpose microprocessor or microcontroller, a digital signal processing (DSP) processor, an integrated circuit, a field programmable gate array (FPGA), a reconfigurable processor, a programmable read-only memory (PROM), or any combination thereof.

The memory 404 can include a suitable combination of any type of computer-readable memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each I/O interface 406 enables the computer 400 to interconnect with one or more input devices, such as the mixer truck's sensors, or with one or more output devices such as a graphical user interface, a memory system and/or a telecommunications network.

Each I/O interface 406 enables the on-board computer 14 to communicate with other components, to exchange data with other components, to access and connect to network resources, to server applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

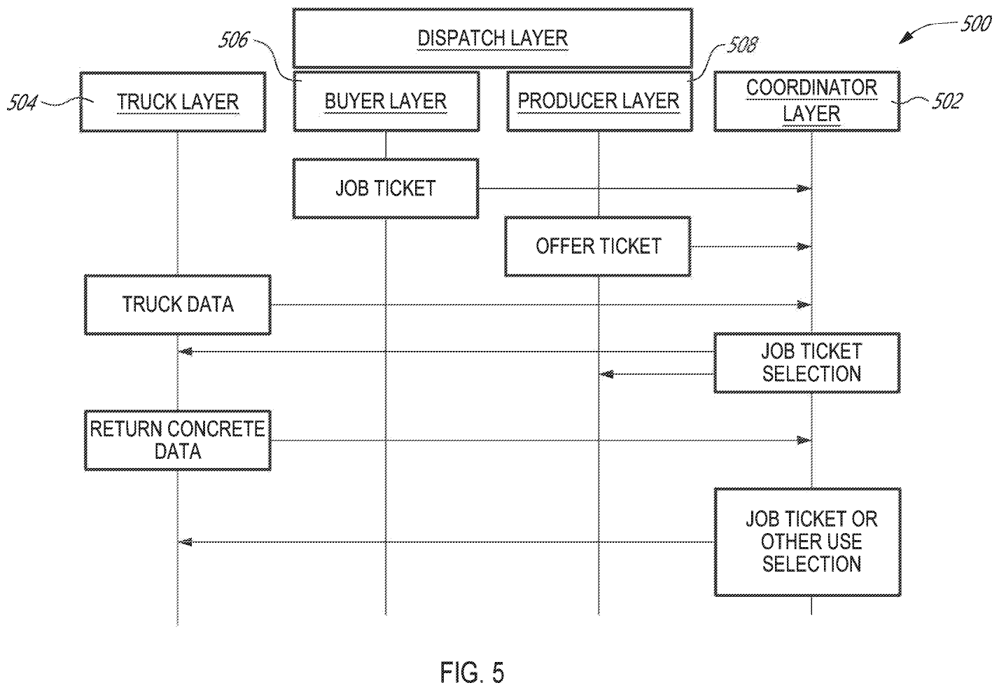

Referring now to FIG. 5, a schematic diagram 500 showing example communication paths between the software layers is shown. As shown, a coordinator layer 502 is communicatively coupled to a truck layer 504, a buyer layer 506 and/or a producer layer 508. The arrows generally represent example queries or communications occurring between the different layers at high speed during a working day. Any one of the coordinator layer 502, the truck layer 504, the buyer layer 506 and the producer layer 508 can be operated by one or more computers such as the one shown in FIG. 4. The layers can be ran by one or more local computers, one or more remote computers and/or on a cloud-based processing platform. In some embodiments, each layer is stored on the memory and accessible by the processor of a corresponding computer such as computer 400.

The computer 400 and the software layers 502, 504, 506 and 508 described above are meant to be examples only. Other suitable embodiments of the on-board computer 14 can also be provided, as it will be apparent to the skilled reader.

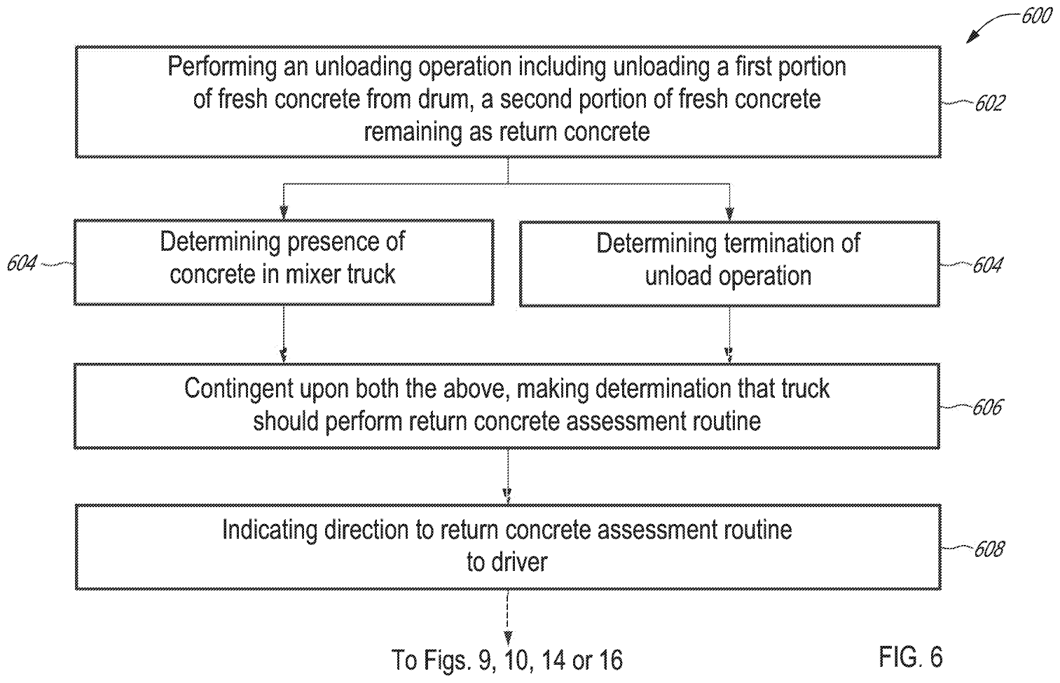

FIG. 6 shows a flowchart of a method 600 including some steps for instructing a concrete mixer truck having with return concrete in accordance with one embodiment.

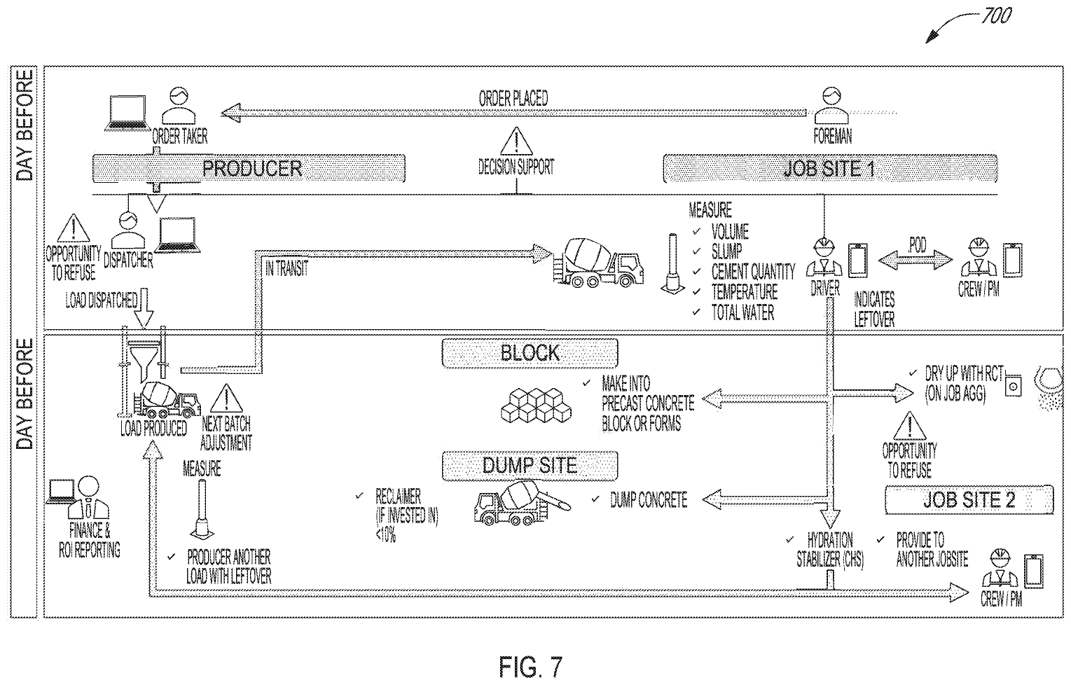

The concrete mixer truck can be a concrete mixer truck such as exemplified in FIG. 1, for instance, which, following a match between an offer ticket and a job ticket, is directed to a batch plant to receive a load of concrete, such as presented in the method 700 of FIG. 7. Upon loading, batch data including details about the quantity and composition of the load of concrete actually loaded into the truck are communicated to the coordinator layer by the batch plant, the truck is then being directed to the job site to unload. The fresh concrete can continue to evolve in the truck over time during transit, in terms of measurands such as slump, temperature, added water content, etc., and sometimes water and/or additives can be added before reaching the job site. The truck can be equipped in a manner to allow to precisely track water and/or additive addition into the batch in some embodiments, in a manner to continue to enable precisely defining the nature of the fresh concrete which was unloaded at the job site. In some cases, only a first portion of the fresh concrete is unloaded during the unloading operation at the job site, a second portion of leftover concrete remaining in the drum and forming what will be referred to herein as “return concrete, ” which is where the method 600 of FIG. 6 begins.

As evoked above, there can be several potential “use scenarios” for return concrete. Some of example use scenarios are presented in the method 700 of FIG. 7. Perhaps the simplest use scenario, which can also be the one which brings the less value out of the fresh concrete, consists in disposing of the return concrete, by dumping it. If the dumping occurs at a dump site, the dump site will typically charge a fee for the dumping, so the return concrete can entail a negative value. Another dumping scenario can involve dumping the return concrete at the job site, which may be free for instance. Another potential use scenario is to use the return concrete to form precast concrete blocks or forms. Such a potential use scenario can allow to reclaim more value than the former one in some cases, since the precast blocks or forms may be sold. Determining whether the precast forming scenario is available or not may require determining whether any moulds are available and empty, and may also require determining whether the available return concrete satisfies any requirements which may exist in association with the demand for precast products. Such requirements can be imposed by precast product customers or by regulations, for instance. Another potential use scenario can consist in adding a chemical formulation into the fresh concrete to break down the fresh concrete chemistry and essentially transforming the return concrete into aggregate, which may then be re-used as ingredients of a future batch of fresh concrete, as road base or otherwise. Determining whether such a potential use scenario is available may require determining whether there is a need or available space for aggregate, whether amounts of the require chemical formulation is available, whether the recipe or nature of the return concrete is suitable for transforming into aggregate, etc.

Additional use scenarios can be considered re-use scenarios, and may entrain a higher value or otherwise be preferable to the preceding use scenarios in some embodiments. Re-use scenarios can involve maintaining the fresh concrete “fresh” until it is used as fresh concrete at another job site, which may require introducing an additive such as a hydration stabilizer when the re-use is expected to take place in excess of a given amount of time, and even re-mixing a limited proportion of the return concrete with fresh ingredients to form a new batch. Determining whether a re-use scenario is available can be significantly more complicated than determining whether the preceding use scenarios are available in some embodiments. For instance, it is common for regulations to be in place to govern the re-use of fresh concrete leftover from one job site at another job site, when the jurisdiction in questions allows it at all. Such regulations depend on the jurisdiction, and can impose various conditions on the re-use of the fresh concrete. Such conditions can include limits of time (age), limits of temperature, limits of proportions into which re-use concrete is mixed in with non-re-use concrete, etc. National regulations in the United States exist in the form of ASTM standards, for instance, which impose specificities on each one of the aforementioned conditions. Time limitations, in particular, impose the additional challenge of making the determination quickly, which can be particularly a challenge when the determination is complicated to make, such as requiring to satisfy a plurality of requirements, especially in cases where information from more than one of the parties involved is required to make the determination, which will typically be the case to determine whether the ASTM standard is satisfied. Additional complexity in making the determination stems from the evolution, in real time, of the potential re-use sites, such as the continuous evolution of job tickets, the requirement of establishing a match, or compatibility, between the return concrete and the job tickets, in an operation which can be similar to establishing a match between an offer ticket and a job ticket but further subject to a more stringent limitation of time, and addressing any additional requirements the job tickets may have specifically for re-use. For instance, some customers or job sites may not accept re-use concrete at all, which may additionally need to be taken into consideration for the determination. Finally, an additional factor which may need to be taken into consideration is transit time associated to a given job ticket. The transit time can be an estimated amount of time for the truck to reach the re-use job site directly where it may, for instance, be mixed into fresh concrete from other trucks, or an estimated amount of time for the truck to reach the re-use job site by first returning to the batch plant to mix in the re-use concrete with new ingredients into a new batch including a certain proportion of return concrete, for example.

As will be understood from the above, determining whether a given use scenario is available for the return concrete can represent certain complexities, especially for re-use scenarios, and the passing of time and unavoidable increase in temperature can be significant factors which would prevent a given batch of return concrete to be successfully matched with a job ticket, again, especially for re-use scenarios. Technologies which allow to make a determination of available use scenarios quickly, facilitate a quick decision-making process, and/or facilitate the communications of instructions quickly to a driver can thus be key in increasing value of return concrete or otherwise making its re-use more frequent.

The method 600 presented in FIG. 6 presents a first technology which can be useful from the point of view of addressing occurrences of return concrete stemming from a partial unload of fresh concrete at a first job site. This technology can help in the general endeavour of providing a quick indication that return concrete is available, which can be required in the process of facilitating a quick decision-making process for instance. This first technology can involve generating a preliminary indication that return concrete is available contingent upon both i) a determination that concrete is present in the mixer truck and ii) a determination that the unload operation has terminated. More specifically, the method 600 includes a step 602 of performing an unloading operation including unloading a first portion of fresh concrete from the drum, with a second portion of fresh concrete remaining as return concrete. The method 600 has steps 604 which can, in parallel. determine a presence of concrete in the mixer truck and determine termination of an unload operation. At step 606, the method 600 includes, contingent upon both steps 604, making a determination that the truck should perform a return concrete assessment routine. At step 608, the method 600 includes a step of indicating direction(s) to return concrete assessment routine to the driver. While either one or both of these determinations can be based on an input from a human, such as the truck driver for instance, automation of the process can help in reducing or entirely removing the likelihood of human error and increasing the speed at which this preliminary indication can be made. There are different ways in which either one of these determination can be automated, in a manner to allow the preliminary indication that return concrete is available to be computer-implemented by an on-board computer, associated directly to the truck carrying the return concrete (such as a computer provided as equipment of the truck and/or a computer carried by the truck driver). Generating this preliminary indication in a computer-implemented manner by an on-board computer can be preferable over other options involving performing this preliminary indication by a remote computer. Indeed, as we will see below, the preliminary indication may need to be communicated to the truck/truck driver, in the form of an instruction to perform a return concrete assessment routine, and there may be no quicker way to have such an instruction communicated to a truck driver than by generating the preliminary indication by a computer having a user interface located in the immediate vicinity of the truck driver. Moreover, as we will see, many ways of making either one, or both of the upstream determinations exist in relation with the truck itself. Performing the preliminary indication in a computer-implemented manner by an on-board computer can also be a means to make the process presented in FIG. 6 independent from any other party, thereby potentially achieving greater reliability than if another party was involved.

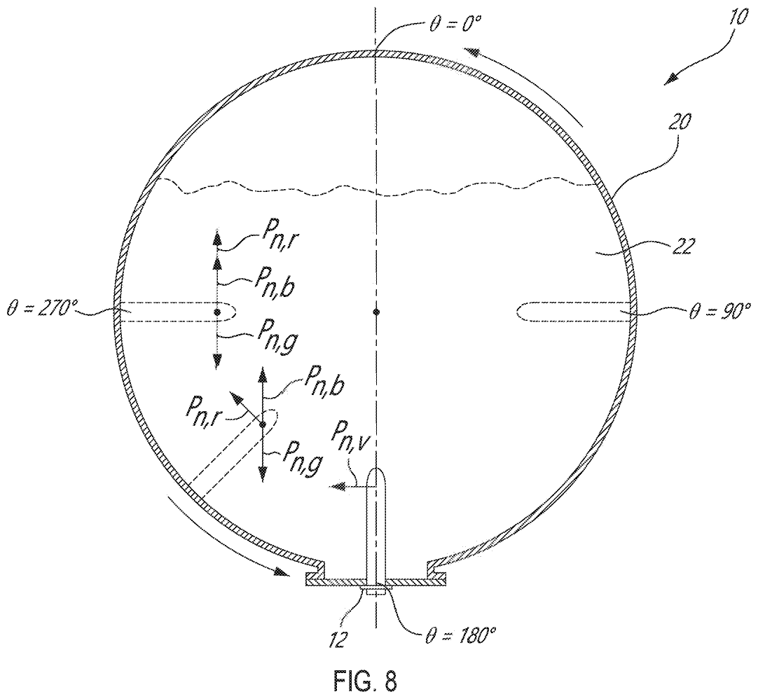

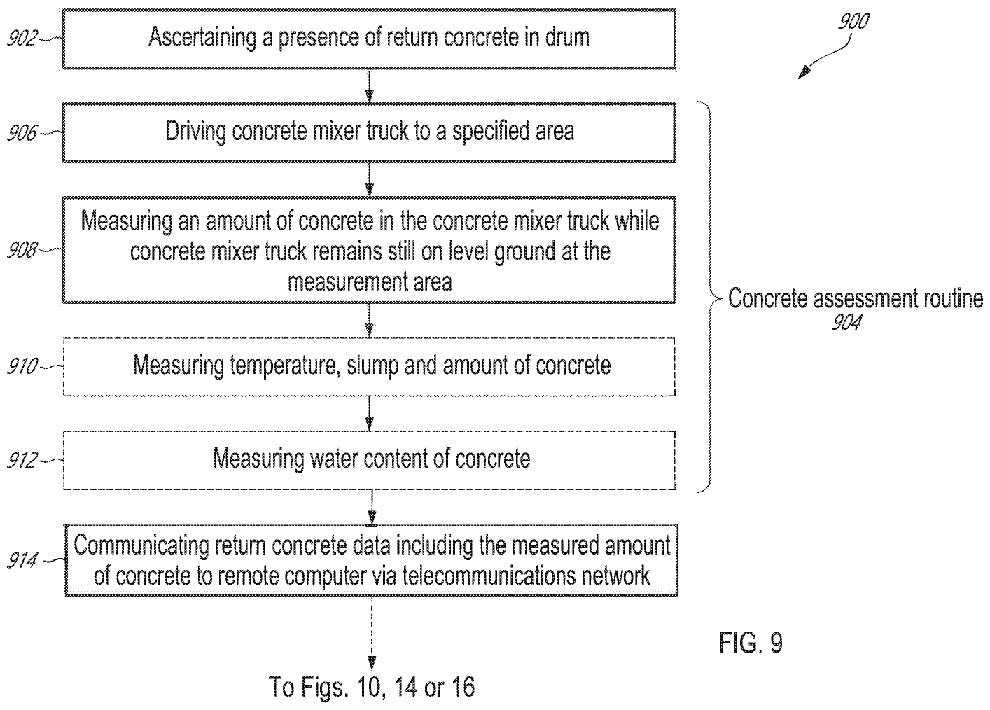

Determining presence of concrete in the mixer truck can be automated when the truck is instrumented by one or more appropriate sensor. For instance, a concrete mixer truck 10 such as presented in FIG. 1 can be equipped with a probe 12, such as perhaps best viewed in FIG. 8. In this example. the probe 12 has a base, which is affixed to the interior wall of the drum 20 of the mixer truck 10. For instance, the probe 12 can be mounted in the inspection door of the drum 20 and the base can have an openable bottom exposed outside the container for operations such as maintenance. During use, the probe 12 rotates with the drum 20 in the rotating direction shown by the arrows, or in the opposite direction, depending of whether the mixer is mixing or emptying the load, for instance. In both cases, the concrete 22 remains toward the bottom of the container due to the action of gravity and its limited viscosity. The probe 12 is thus immersed into the concrete 22 at each revolution and travels therein. The concrete 22 exerts a resistance pressure shown schematically with arrows opposing the movement of the probe. The probe can directly measure parameters such as the position of the probe, the force (or resistance pressure exerted by the substance on the probe), the temperature, etc. In some embodiments, such a probe can be equipped with a load cell, for instance, and adapted to measure the forces between the probe 12 and concrete 22 in the drum 20 in the direction of rotation, as the probe 12 engages and moves through the concrete 22 which remains at the bottom of the drum 20 under the effect of gravity. The probe 12 can subsequently use these parameters to determine the speed, and thence use speed and force values for instance to obtain an indication of properties of the concrete 22 such as the viscosity, the yield, the slump, the cohesion, etc. The probe 12 can be made of any suitable material given the potentially harsh environment. Many examples of such a probe 12 can be provided including, but not limited to, the probe described in the PCT Application Publication No. WO2011/042880, the contents of which are hereby incorporated by reference. With such a probe, a rough rotation speed can be estimated without a position sensor simply by timing the delay between two subsequent substantial increases or decreases in force, which gives an indication of the time it takes for the drum to make a complete revolution. If the information concerning the path of the probe is available (typically linked to the diameter of the truck). the length of the path can be divided by the time to give a rough average speed approximation. Such processing by the processing unit can thus constitute a speed sensor which can replace computation of position data if desired. Similarly, the determination of the presence of the probe in the mixture can be made, for example, with the temperature detected by the temperature sensor. When the temperature increases substantially, the probe is determined to be in the mixture and when the temperature decreases substantially, the probe is determined to have exited the concrete or vice versa, depending on the qualities of the concrete being mixed. By using the force value or the temperature value, the processing unit of the probe and/or the on-board computer are able to determine if the probe is in the concrete without knowing the amount of mixture in the mixer and the determination is therefore independent of the amount of mixture in the mixer.

As such, the probe when combined with means to determine its angular position around its circular movement path with the drum, can allow detecting an entry point (angle) and an exit point (angle) of the probe in the concrete. Simply detecting an entry point or an exit point, or a force in excess of a certain threshold anywhere in between, may be suitable for generating a determination that concrete is present in the drum. Such a probe can also be used to sense a direction of rotation of the drum since the orientation of the sensed force can revert when the direction of rotation is reversed. Other ways of automating the detection of the presence of concrete in the mixer truck with a suitable sensor or combination of sensors can be suitable as well. For instance, in many cases, the truck's computer will know how much quantity of concrete was loaded into its drum at the batch plant, and may further be enabled to know approximately how much quantity of fresh concrete is unloaded for a given number of rotations of the drum in the unload direction. If the mixer truck is equipped with any suitable sensor for determining a number of rotations, and an orientation of the rotations which may be the case for one or more of a probe such as presented above, an accelerometer, or a gyroscope associated to the drum for instance, and even perhaps of a hydraulic pressure sensor associated to the hydraulic motor generating the rotation of the drum, to name a few examples, the number of rotations can be counted. Detection of a mismatch between the approximate quantity of unloaded concrete and the initial amount of concrete, say in excess of a given threshold, can be the source of an automated determination that return concrete is available in the concrete mixer truck.

Determining termination of the unload operation can also be automated when the truck is instrumented by one or more appropriate sensor. For instance, detecting a change in the direction of rotation from the unloading direction to the mixing direction, which can be detected using one or more sensors as presented above, can be used as a basis for the determination of termination of the unload operation. If the loading operation has previously been detected, e.g., by detecting that the rotation direction is in the unload direction, or by any other suitable means, simply detecting that the rotation direction is in the mixing direction can be sufficient to make the determination. Similarly, detecting movement of the truck, such as via a truck speed signal for instance, following a detected unload operation can be used as a basis to make the determination. Using a Global Positioning System (GPS) or other location sensor, can also form a basis to make the determination. For instance, a GPS functionality of the truck's computer can detect that the truck has reached the job site. Subsequent departure of the truck from the job site, which can also be detected by the GPS functionality of other suitable means, can then form the basis for the determination. A sensor can be mounted to a wheel of the truck and generate a signal indicative of a rotation of the wheel, which can be interpreted in terms of movement of the truck, for instance. A hydraulic pressure sensor may also provide a signal which can be used in making the determination. In some embodiments, it may be preferred for the truck to receive a communication originating outside the truck to make the determination, such as a job site operator input, dispatcher input interpreting job site video footage, or job site geofence to name a few examples, but as presented above, there can be advantages on relying on on-truck functionalities in making an automated determination at least in some embodiments.

The preliminary indication of return concrete can amount to applying an AND gate to both above-mentioned determinations in some embodiments. An on-board computer can use the preliminary indication of return concrete to trigger a function of indicating this condition to the driver. The indication to the driver can be automated in various forms depending on the embodiment. For instance, it can be a visual indicator such as triggering an illuminated indicator present on the dashboard of the truck, it can be a more elaborate visual indicator embodied in a graphical user interface of a display screen of a truck or of a driver device. It can also be a message, such as SMS or MMS or an email for instance, sent by a truck computer to a driver device. The indicator can also be audible for instance, such as a buzzing sound or synthetic voice message, to name still other examples. In all these examples, the indicator is perceivable by the driver of the concrete mixer truck.

In some embodiments, the preliminary indication of return concrete can be communicated externally from the truck, such as to a remote computer via a telecommunications network. Such a remote computer can have a receiver software functionality forming part of the coordination layer for instance. However, in many embodiments, the preliminary indication, though helpful, will not be sufficient in and of itself to allow matching the occurrence of available return concrete to one or more re-use scenarios. In particular, job ticket specifications, and/or regulations, may impose requirements associated to information in excess of the preliminary indication of return concrete. Indeed, such information in excess of the preliminary indication of return concrete may need to be acquired or otherwise communicated in the form of return concrete data by an on-board computer to a remote computer over a telecommunications network to enable the determination of whether or not a given occurrence of return concrete can be reused in association with another job ticket. A portion of the return concrete data may be collected and communicated by the truck itself, whereas another portion of the concrete data may be collected and communicated by another computer. We will look into the subject of return concrete data acquisition by the truck first before returning to the subject of return concrete which can be acquired from another source, such as batch data for instance, later in this specification.

The nature of the return concrete data which can or must be acquired or communicated by the truck itself (which can be referred to as truck data or return concrete data) can depend on the specificities of the embodiment. It can be common to many embodiments, for instance, to require more precise measurements of the quantity of return concrete in the drum, than a simple preliminary indication that return concrete is available. In particular, regulations can impose quantity-related restrictions or conditions, such as measuring the quantity of return concrete within a certain determined tolerance value, or imposing a maximum proportion or return concrete to non-return concrete to be mixed together as a condition for re-use. Moreover, job ticket requirements and/or matching requirements can impose quantity-related restrictions or conditions. For instance, to agree to pay a certain amount for a batch including re-use concrete, the customer may require a relatively precise measurement of the quantity of return concrete. To agree to match a batch including re-use concrete with a job ticket, a dispatcher may require a certain minimum quantity of concrete, or a match between a quantity of concrete in the job ticket and the quantity of re-use concrete.

Satisfying any or all of these quantity-related restrictions or conditions may require a second technology involving performing a concrete assessment routine, an example of which is presented in the flowchart of method 900 in FIG. 9, and which can be used in combination or independently from the technology presented in relation with the method 600 of FIG. 6. More specifically, the method 900 includes a step 902 of ascertaining a presence of return concrete in the drum. The method 900 includes a concrete assessment routine 904 including a step 906 of driving the concrete mixer truck to a specified area, a step 908 of measuring an amount of concrete in the concrete mixer truck while the concrete mixer truck remains still on level ground at the measurement area, a step 910 of measuring temperature, slump and an amount of concrete within the drum, a step 912 of measuring water content of the concrete. Steps 910 and 912 can be optional in some embodiments. The method 900 includes a step 914 of communicating return concrete data including the measured amount of concrete to one or more remote computers via one or more telecommunications networks.

In this example, the concrete assessment routine 904 includes performing a measurement of the return concrete while the concrete mixer truck remains still on level ground, which is the case for both of the two following example return concrete quantity measurement techniques. The first one of these techniques can involve the measurement of volume using a probe or sensor such as described above. For instance, the volume of return concrete can be determined using the in-drum probe. More specifically, the position at which the probe enters and exits the concrete during a rotation of the drum can be identified by detecting for instance a sudden increase and a sudden decrease in the force value measured by the load cell. Given a known geometry of the drum, a value indicative of the volume of the return concrete contained in the drum can be determined. Indeed, for such a measurement to be considered sufficiently precise, it may be required for the measurement to take place when the truck is still and on level ground. The second one of these techniques can be to use a truck weighing scale which may be provided at the job site or batch plant, for instance. A truck weighing scale also has a flat, level surface and requires the truck to remain still while the weighing is being performed. If using a weighing scale, the truck weight can be wirelessly communicated to the truck system to achieve a fully automated operation, or alternately displayed to and then manually entered by the truck driver via an interface of an on-board computer. If, such as in the United States at the time of drafting this specification, the regulations require the measurement to be made in units of volume, software can be used to perform a conversion of weight to volume either on the truck or in a remote computer to which the relevant data has been communicated to, which can require a measurement of density of the return concrete. The density of the concrete can be measured or calculated using one or more systems and techniques. For instance, in one of these techniques, the probe measures a first pressure value indicative of a normal pressure exerted on the probe by the fresh concrete at a first circumferential position of the drum during rotation of the drum, the probe measures a second pressure value indicative of a normal pressure exerted on the probe by the fresh concrete at a second circumferential position during rotation of the drum, the first circumferential position being different from the second circumferential position; and a processing device determines a density value of the fresh concrete based on the volume of the probe and on a difference between the first pressure value and the second pressure value. In another one of these techniques, the density is determined by measured the time required for an acoustic signal to travel across a sample of concrete and by comparing the required time (or the speed of sound of the acoustic signal if the distance of propagation of the acoustic signal across the concrete is known) to reference data. Once acquired, the measure quantity can be stored in computer memory as part of truck data and/or communicated via a telecommunications network. In cases where the concrete assessment routine requires measuring a quantity of concrete while the truck is still and on level ground, the truck may need to be driven to a suitable location of level ground before the quantity can be measured. Referring to the explanations presented above, in one embodiment, the indication to the driver can serve as an instruction or command instructing the driver to drive the truck to the suitable location, which can be referred to as the measurement location, a step which can be performed by the truck driver's cooperation, for instance, or via an automated driving sequence. The indication to the driver can also serve as an instruction instructing the driver to perform any and all act required by the concrete assessment routine, if any such acts is required.

Various restrictions or conditions other than quantity-related restrictions or conditions may exist, and additional data may need to be acquired via sensors of the truck. For instance, regulations can impose limits on the temperature of the return concrete, which can in some cases be known to somewhat predictively increase over time, limits on the amount of water in the return concrete, and/or limits on the amount of additive in the return concrete, etc., any and all of which may be acquired by suitable sensors equipping the truck in a manner to be stored into memory as part of return concrete data. For instance, in some embodiments, water content can be measured by an on-truck water measuring device or using a technique involving, when water is added to the concrete, obtaining a quantity and a thermal capacity of the concrete in the mixer drum prior to the addition, obtaining a temperature of said water added to the mixer drum, calculating the amount of water added to the mixer drum based on said quantity and thermal capacity of the concrete and the temperature of the added water. An example of such a technique is described in the PCT Patent Application Publication No. WO2014/071526, the contents of which are hereby incorporated by reference. The exact selection of values to be included as return concrete data may vary from one embodiment to another, as a function of the specificities of the embodiment in question.

The process of acquiring or retrieving return concrete data via the truck systems for the purpose of facilitating the establishment of a match between the return concrete and a job ticket can be referred to as a concrete assessment routine. The concrete assessment routine can include acquiring data via sensors of the truck, such as the quantity of return concrete measurement, slump, temperature measurement, water content measurement and/or any other suitable measurement. The concrete assessment routine can be partially or fully automated and performed by an on-board computer, and in addition to acquiring sensor signal(s), the concrete assessment routine can further involve retrieving return concrete data which has been previously stored in a memory readable or otherwise forming part of the computer. For instance, batch data including information associated to the initial recipe (e.g., quantity of cement, type or size of aggregate, strength) may have been previously made available for storing as data in the memory, and it may be convenient to retrieve this data and include it together with data stemming from immediate or otherwise more recent sensor input. Moreover, information concerning a quantity of a given, or of any material added into the fresh concrete since batching can be stored into the memory as data at the time when it was added. This can include, for instance a quantity of water added at one time, or at more than one time subsequently to the loading operation. This can include, for instance, a quantity of one or more additive or admixture added at one time, or at more than one time subsequently to the loading operation. The concrete assessment routine can thus involve acquiring and/or collecting any and all suitable truck data to form part of the return concrete data.

The acquired and/or collected truck data can then be communicated by an on-board computer to a remote computer via a telecommunications network, in the objective of allowing a match to be made between the return concrete and an open job ticket.

As presented above, limiting the amount of time between the termination of the unload operation and the communication of the return concrete data by the truck to the remote computer can be a key factor in increasing the rate at which return concrete loads are successfully matched with job tickets for re-use. In some embodiments, it can be preferred for this amount of time to be of under 30 minutes, and in some other embodiments, it can be preferred for this amount of time to be of under 5 minutes. It will be understood that partially if not fully automating the process spanning from the beginning of the method 600 of FIG. 6 to the end of the method 900 of FIG. 9 with the use of a computer can be essential to achieve such levels of performance.

It will also be noted that it may be useful to update some, or all of the truck data elements over time, subsequently to the initial communication of the return concrete data. This can be achieved by acquiring new values via one or more sensors or input means for instance, via the computer, and the computer performing one or more subsequent transmissions over the telecommunications network, for instance. This may be relevant in particular for values such as temperature or slump of the return concrete for instance. It may also be suitable in some embodiments to update the return concrete data when one or more specific events occur, such as when the truck detects that it has arrived at the batch plant (typically a default action subsequently to the unloading operation and concrete assessment routine), nature and quantity of admixture or additive addition, quantity of water addition, etc.

The technologies described above can be used in addition to, or independently from the technologies described below. In any case, the technologies described in the following paragraphs can help in the general endeavour of providing a quick indication that return concrete meeting applicable regulations is available, a quick indication of a list of eligible job tickets for a given return concrete load and/or a list of eligible return concrete loads for a given job ticket, which can all be required for facilitating a quick decision-making process for instance. There are presented systems and methods for handling mixer trucks containing return concrete loads. The systems and methods can be operated by one or more software connectivity layers directly or indirectly commutatively coupled to one another. For instance, the software connectivity layers can include, but are not limited to, a coordinator layer, a producer layer, a buyer layer, and a truck layer.

In some embodiments, the steps are performed by the coordinator layer which is communicatively coupled to the producer layer, the buyer layer and the truck layer. In some embodiments, the coordinator layer can be remote from the buyer(s) or associated job site(s), remote from the producer(s) and remote from the truck(s). For instance, in some embodiments, the coordinator layer is provided in the form of hardware and software components that are part of a cloud-based processor platform and the like. Accordingly, the coordinator layer can receive, store and/or index information incoming in a real time or quasi-real-time basis for quick retrieval from the producer layer, the buyer layer and/or the truck layer.

Although a given piece of information can be communicated directly between the truck layer and the coordinator layer in some embodiments, any given piece of information can be communicated indirectly between the truck layer and the coordinator layer via the producer layer, for instance or via the buyer layer. There can also have redundancy in that the coordinator layer can receive a given piece of information independently from two or more software connectivity layers.

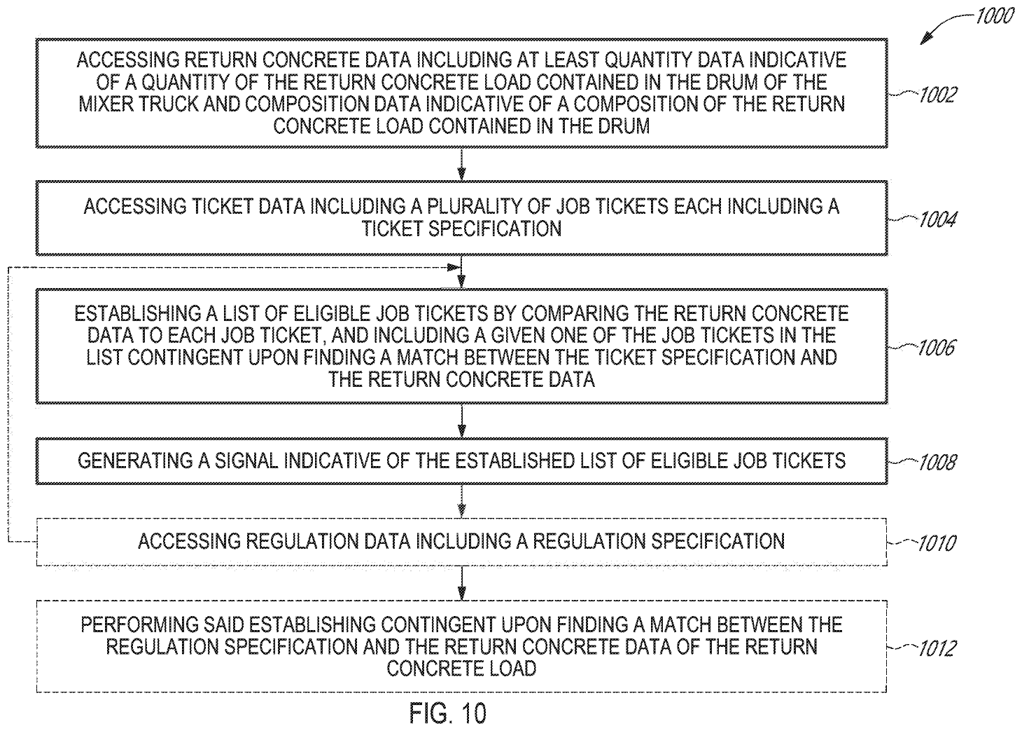

FIG. 10 is a flow chart of an example of a computer-implemented method 1000 for handling a mixer truck containing a return concrete load. The method can be operated by one or more software connectivity layers directly or indirectly commutatively coupled to one another. For instance, the software connectivity layers can include, but are not limited to, a coordinator layer, a producer layer, a buyer layer, and a truck layer. Although FIG. 10 is described from the point of view of the coordinator layer which performs each method steps of the method, the method could be viewed from any other suitable connectivity layer.

At step 1002, the coordinator layer accesses return concrete data associated with the return concrete load contained in the drum of the mixer truck. The return concrete data in this example includes at least quantity data indicative of a quantity (e.g., a volume, a weight) of the return concrete load contained in the drum of the mixer truck and composition data indicative of a composition of the return concrete load contained in the drum. The return concrete data can be measured according to the techniques discussed above. In some embodiments, the quantity data is received as part of truck data from the truck layer. However, the quantity data can be received from the producer layer in some embodiments. The composition data can be received from the producer layer, from the buyer layer and/or from the truck layer, depending on the embodiment.

At step 1004, the coordinator layer accesses ticket data including job tickets each including a ticket specification. The ticket specification can include a minimum quantity requirement and a composition requirement. The ticket data is generally received or communicated from the buyer layer.

At step 1006, the coordinator layer establishes a list of eligible job tickets by comparing the return concrete data to each job ticket. The coordinator layer includes any given one of the job tickets in the list contingent upon finding a match between the ticket specification and the return concrete data.

At step 1008, the coordinator layer generates a signal indicative of the established list of eligible job tickets. The signal generated can trigger one or more actions. For instance, in some embodiments, the signal is used to display the established list of use eligible job tickets on a graphical user interface present in a cabin of the mixer truck, an office of the buyer or producer. Additionally or alternatively, the method can include a step of communicating the established list of eligible job tickets over an accessible network and/or storing the established list of eligible job tickets on an accessible memory. When displayed on a graphical user interface accessible by the truck driver and/or a dispatch, the established list of eligible job tickets can offer a quick glance at the eligible job tickets for a given return concrete load, which may in turn help to timely trigger actions to re-use the return concrete load to maximize value but also to reduce waste.

In some embodiments the coordinator layer accesses regulation data including a regulation specification, and performs, at step 1010, a step of establishing the list of eligible job tickets contingent upon finding a match between the regulation specification and the return concrete data of the return concrete load. For instance, if a given return concrete load is deemed to be too old, and/or too warm, the given return concrete load may go rogue and not appear on the established list to avoid confusion on the truck driver and/or dispatch side. In some embodiments, the step 1010 can be performed prior to the step 1008 of generating the signal indicative of the established list of eligible job tickets. In this way, the generated list may be free of regulatory non-compliant return concrete loads.

It is envisaged that the method can further include a step of selecting a given one of the eligible job ticket via a user input on the graphical user interface for instance, and a subsequent step of removing the selected one of the eligible job tickets from the list. In these embodiments, such a selection may infer that dispatch had decided to attribute a given return concrete load to one of the eligible job tickets, and thereby mark it or remove it off the list for instance to avoid other trucks to be assigned afterwards. As such, one eligible job ticket may be selected only by one of the truck drivers having access to the ticket data. The log of job ticket maintained by the coordinator layer may be updated as eligible job ticket selections are received.

In some embodiments, the return concrete data can include location data indicative of a location of the return concrete contained in the drum of the mixer truck and the job tickets can include a corresponding job site location. The location can be provided in the form of GPS location, postal address location and the like. As such, in determining which job ticket is eligible, the method can perform a step of determining whether a match is found between the job site location and the location of the return concrete load, and add the matching job tickets on the list. The eligible job tickets may be determined upon finding that a travel time from the location of the mixer truck to the location of the job site is below given travel time threshold. The travel time threshold can be dictated by the applicable regulations.

In some embodiments, the return concrete data can further include concrete parameter data indicative of at least another parameter of the return concrete contained in the drum of the mixer truck. In these embodiments, each job ticket can further include a requirement for the other parameter. As such, the list of eligible job tickets can include the given one of the job tickets in the list contingent upon finding an additional match between the requirement for the other parameter and the parameter data of the return concrete data. In some embodiments, the other parameter is added water content, temperature, concrete strength, aggregate size, slump and/or a combination thereof.

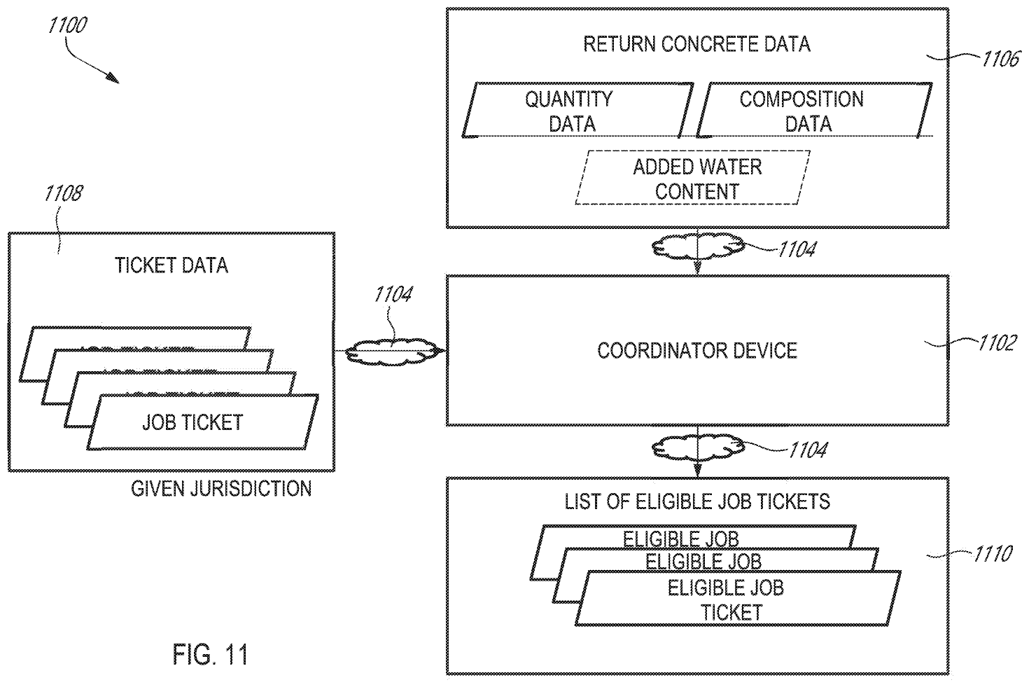

FIG. 11 shows a block diagram of an example of a system 1100 for handling a mixer truck containing a return concrete load. As shown, the system has a coordinator layer which can be communicatively coupled to other connectivity layers such as the buyer layer, the truck layer and the producer layer via one or more internal networks, external networks and/or public telecommunications networks. In this example, the coordinator layer can be provided in the form of a coordinator device 1102 communicatively coupled to an external network 1104. The coordinator device 1102 of this example can be partially or wholly cloud-based and/or on-premise and has a processor and a memory having stored thereon instructions that when executed by the processor perform the steps of the method 1000 of FIG. 10.

As shown, the coordinator device 1102 accesses return concrete data 1106 including at least quantity data indicative of a quantity of the return concrete load contained in the drum of the mixer truck and composition data indicative of a composition of the return concrete load contained in the drum. As depicted, the return concrete load includes added water content as well.

The coordinator device 1102 accesses ticket data 1108 including a job tickets each including a ticket specification. The ticket specification can include a quantity requirement, a composition requirement, a minimum added water content requirement and the like.

Upon access to proper data, the coordinator device 1102 establishes a list of eligible job tickets 1110 by comparing the return concrete data 1106 to each job ticket, and includes a given one of the job tickets in the list contingent upon finding a match between the ticket specification and the return concrete data 1106. For instance, the coordinator layer may find a match between the quantity requirement and the quantity data of the return concrete data 1106, a match between the composition requirement and the composition data of the return concrete data 1106, and/or a match between the minimum added water content to the water content data of the return concrete data 1106.

Once the list of eligible job tickets 1110 is established, the communicator device can generate a signal indicative of the established list of eligible job tickets. The signal can be communicated to the truck layer, to the buyer layer, to the producer layer, or a combination thereof, to name a few examples. In some examples, the signal can be used to generate an alert or a prompt window on a graphical user interface which indicates which eligible job tickets have been found for a particular return concrete load. In some embodiments, the coordinator layer selects one of the eligible job tickets and attributes it to the mixer truck, preferably with no human interaction. In this way, a driver of the mixer truck has a minimal amount of material work to do in the selection of the right eligible job ticket, thereby reducing risks of human errors in the management of the return concrete load, which may in turn maximize value and reduce waste.

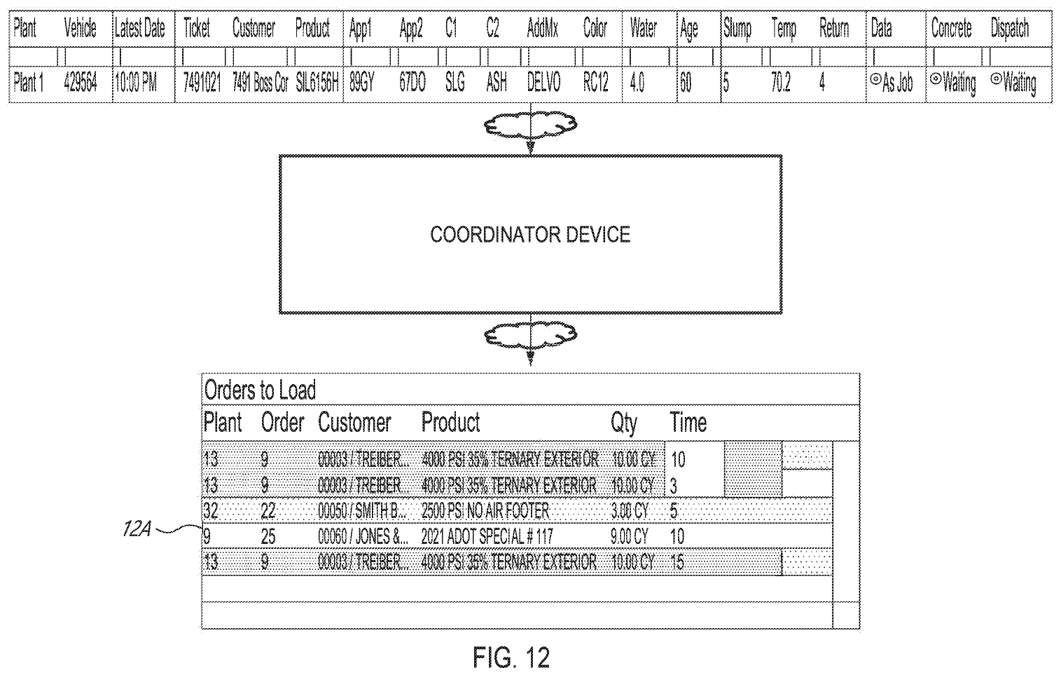

FIG. 12 shows an exemplary return concrete data incorporating information regarding a return concrete load. For instance, the information can include, but are not limited to, a quantity, a composition, an elapsed time since batching, an added water content, a temperature, additive treatment information and the like. The information can stem from the buyer layer, the truck layer and/or the producer layer, depending on the embodiment. The information can be presented in the form of a data array or list. As shown, the coordinator device accesses the return concrete data and, considering a list of available job tickets, matches the return concrete data to data of the available job tickets to establish a list of eligible job tickets, an example of which is shown at inset 12A.

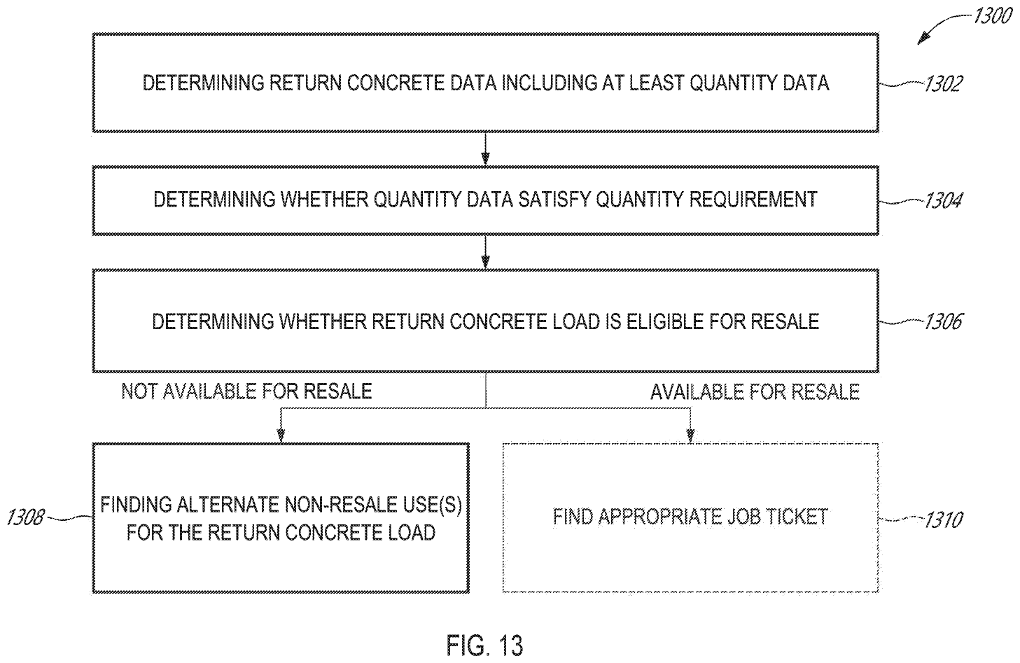

FIG. 13 shows an example of a flow chart of a method 1300 for handling a return concrete load. As shown, the method includes a first routine 1302 for determining return concrete data including at least quantity data indicative of a quantity of the return concrete load contained in the drum of the mixer truck. Another routine 1304 is provided to determine whether the quantity data satisfy quantity requirements. For instance, if the quantity of return concrete remaining in the drum after a partial delivery is below a first quantity threshold, e.g., 2 cubic yards, then the invoicing of the buyer remains unchanged. However, contingent upon the quantity of the return concrete load exceeding the first quantity threshold, the client may be deducted from that unused quantity, in some embodiments.

The method includes a routine 1306 which determines whether the return concrete load is eligible for resale. For instance, the return concrete load may be eligible for resale contingent finding that the return concrete load meet the requirements of the application regulation. For instance, the temperature is below 38 Celsius degrees, the elapsed time since batching is below 90 minutes and/or the return concrete load would amount for no more than 50% of the job ticket. If it is determined that the return concrete load is not eligible for resale, a routine 1308 may be performed in which it is determined whether the return concrete load should be used to form concrete blocks (if empty blocks are available at the producer plant), to reclaim aggregate from the return concrete load (if the quantity is above a given threshold), to donate the return concrete load to possible buyers or otherwise interested parties, or finally to simply dump the return concrete load at a dump site or a job site.

It the method finds that the return concrete load is eligible for resale, the coordinator layer may, at routine 1310, determine if there are job tickets which ticket specification indicates that return concrete is accepted. If such job tickets are found, the coordinator layer can perform a slump verification routine. The slump of the return concrete load can be measured using a rheological probe mounted inside the wall of the drum of the mixer truck or any other suitable sensor. Accordingly, the slump can be part of the return concrete data, and communicated to the coordinator layer from the mixer layer, for instance. If the slump is not acceptable, the return concrete load may be treated in accordance to a concrete stabilization process or other concrete treatment process at the producer plant. Doing so involves instructing the mixer truck carrying the return concrete load to go back to the producer plant so that the return concrete load be conveniently treated. In some embodiments, a return concrete load which is treated according to regulations can be reused for an extended period of time.

If the slump is determined to be within an acceptable slump range, the return concrete load can be marked as active on an established list of eligible return concrete loads. Upon matching the return concrete load to a corresponding job ticket, the mixer truck can be instructed to go to the job site location for the pouring of the return concrete. Once the job ticket is done, the coordinator layer can be reinitiated for the remaining concrete in the drum.

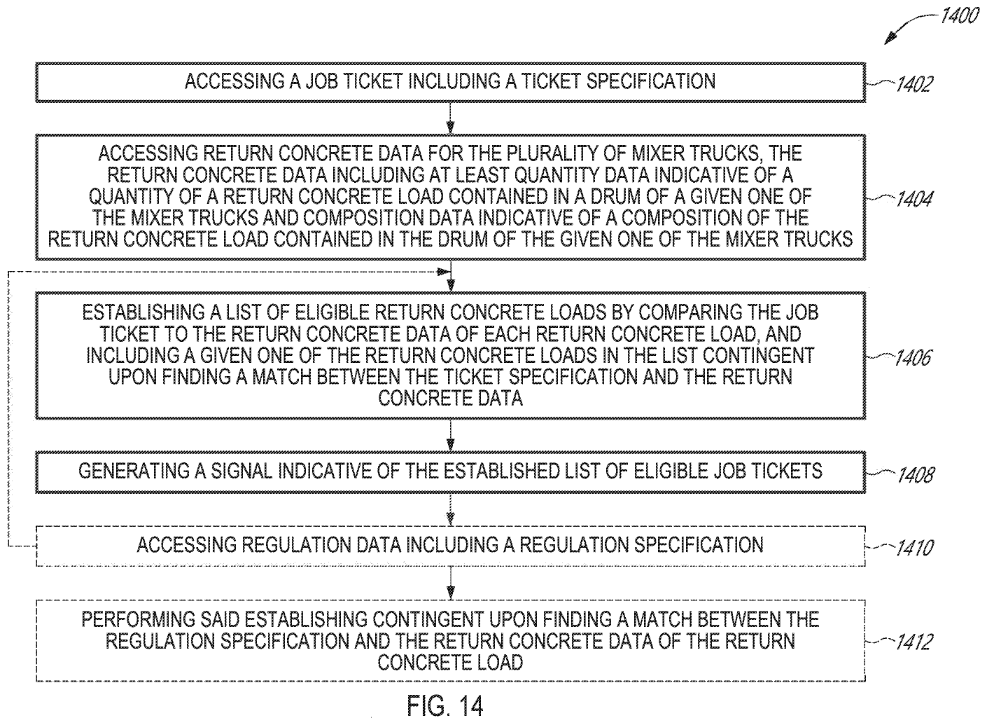

FIG. 14 is a flow chart of an example of a computer-implemented method 1400 for handling a mixer truck containing a return concrete load. The method can be operated by one or more software connectivity layers directly or indirectly commutatively coupled to one another. For instance, the software connectivity layers can include, but are not limited to, a coordinator layer, a producer layer, a buyer layer, and a truck layer. Although FIG. 14 is described from the point of view of the coordinator layer which performs each method steps of the method, the method could be viewed from any other suitable connectivity layer.

At step 1402, the coordinator layer accesses a job ticket including a ticket specification. The ticket specification can differ from an embodiment to another, but can include a quantity requirement, a composition requirement, an added water content requirement, a slump requirement and the like.

At step 1404, the coordinator layer accesses return concrete data associated to return concrete loads contained in the drums of a number of mixer trucks. The return concrete data includes at least quantity data indicative of a quantity of a return concrete load contained in a drum of a given one of the mixer trucks and composition data indicative of a composition of the return concrete load contained in the drum of the given one of the mixer trucks. In some embodiments, the return concrete data can be partially or wholly received from the truck layer, from the buyer layer and/or from the producer layer.

At step 1406, the coordinator layer establishes a list of eligible return concrete loads by comparing the job ticket to the return concrete data of each return concrete load. The list of eligible is such that it includes any given one of the return concrete loads contingent upon finding a match between the ticket specification and the return concrete data.

At step 1408, the coordinator layer generates a signal indicative of the established list of eligible return concrete loads. The established list of use eligible return concrete loads can be displayed on a graphical user interface based on the generated signal. Additionally or alternately, the established list of eligible return concrete loads can be communicated over an accessible network and/or stored on an accessible memory.

It is envisaged that the method can further include a step of selecting a given one of the eligible return concrete loads via a user input for instance, and a subsequent step of removing the selected one of the eligible return concrete loads from the list.

At optional step 1410, the coordinator layer accesses regulation data including a regulation specification, and performs, at step 1412, a step of including a return concrete load into the list contingent upon finding a match between the regulation specification and the return concrete data of the return concrete load. As such, only regulatory safe return concrete loads may be included in the established list. In some embodiments, the step 1410 can be performed prior to the step 1408 of generating the signal indicative of the established list of eligible job tickets. In this way, the list may always be free of non-regulatory accepted return concrete loads.

The return concrete data for each mixer truck can include concrete parameter data indicative of at least another parameter of the return concrete contained in the drum of the corresponding mixer truck. In such embodiments, the job ticket can further include a requirement for the other parameter. The list of eligible return concrete loads includes return concrete loads contingent upon finding an additional match between the requirement for the other parameter and the parameter data of the return concrete data. Examples of such additional parameters can include water content, temperature, concrete strength, aggregate size, and slump, to name a few examples.

The method can include a step of, upon selecting a given one of the eligible return concrete loads, removing the selected one of the eligible return concrete loads from the list. In some embodiments, the list of eligible return concrete loads can include return concrete loads only upon determining that a match between a job site location associated to the job ticket and location data of the mixer truck. The method may also include a step of refreshing the steps 1402 and 1404 and repeating the step 1406 so as to update the established list of eligible return concrete loads in real time or quasi-real time again to maximize value and/or reduced waste.

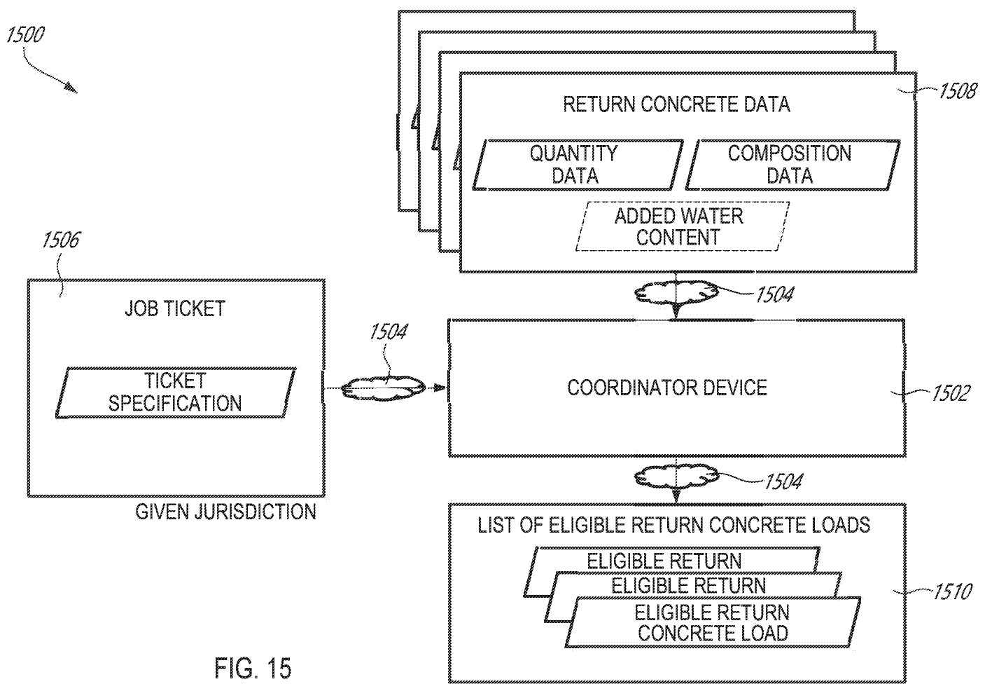

FIG. 15 shows a block diagram of a system 1500 for handling mixer trucks each containing a return concrete load. As shown, the system has a coordinator device 1502 which is communicatively coupled to a truck layer, a buyer layer and a producer layer, or a combination thereof, via a telecommunications network or any other internal or external network 1504. The coordinator device 1502 is configured to access a job ticket 1506 including a ticket specification. The job ticket 1506 can be submitted to the coordinator layer by the customer layer, for instance. The coordinator device 1502 can access return concrete data 1508 for each of a number of mixer trucks. The return concrete data 1508 typically includes at least quantity data indicative of a quantity of a return concrete load contained in a drum of a given one of the mixer trucks and composition data indicative of a composition of the return concrete load contained in the drum of the given one of the mixer trucks. Once the suitable data are accessed, the coordinator device 1502 is configured to establish a list of eligible return concrete loads 1510 by comparing the job ticket 1506 to the return concrete data 1508 of each of the return concrete loads. More specifically, the coordinator device 1502 includes any given one of the return concrete loads in the list contingent upon finding a match between the ticket specification and the return concrete data. Then, a signal may be generated based on the established list of eligible return concrete loads.

It is envisaged that the list of eligible return concrete loads can be displayed on a graphical user interface accessible to a truck driver, a batcher, a dispatch or anybody else performing the coordinator role, for easy and intuitive consultation of the possible return concrete loads eligible for a given job ticket.

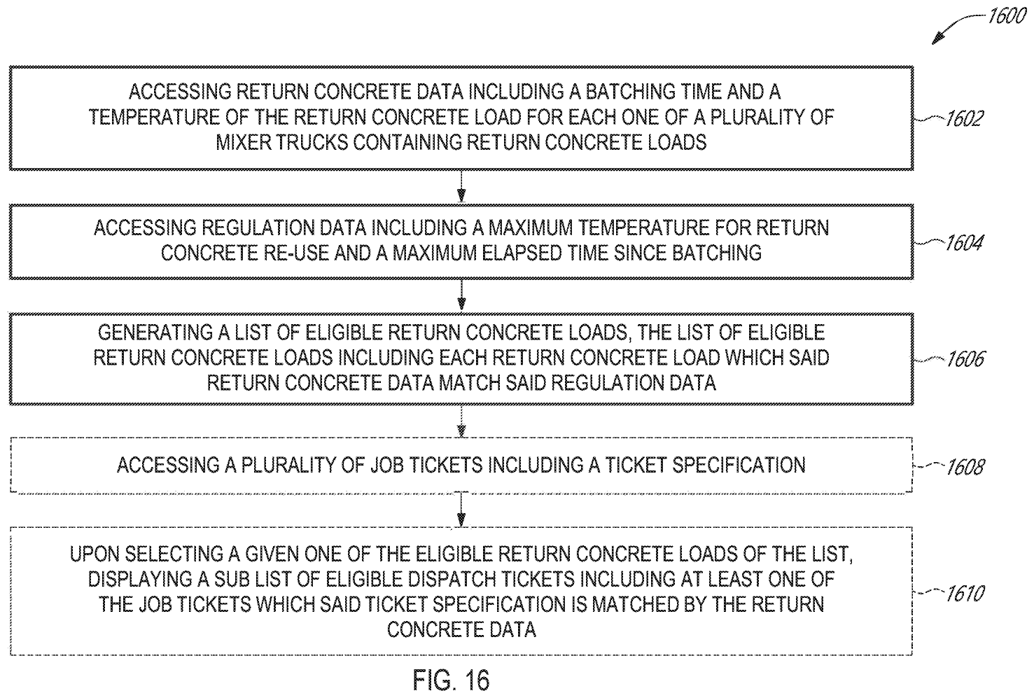

FIG. 16 is a flow chart of an example of a computer-implemented method 1600 for handling mixer trucks containing return concrete loads. The method can be operated by one or more software connectivity layers directly or indirectly commutatively coupled to one another. For instance, the software connectivity layers can include, but are not limited to, a coordinator layer, a producer layer, a buyer layer, and a truck layer. Although FIG. 16 is described from the point of view of the coordinator layer which performs each method steps of the method, the method could be viewed from any other suitable connectivity layer.

At shown, at step 1602, the coordinator layer accesses return concrete data including at least a batching time and a temperature of the return concrete load for each one of a plurality of mixer trucks containing return concrete loads.

In some embodiments, the return concrete data are received from a corresponding one of the mixer trucks, a batching plant, a dispatch facility and an external network such as the Internet. In some embodiments, the coordinator layer can access the batching time associated with a given one of the return concrete loads by retrieving the corresponding batching time on a database made accessible for instance via the producer layer or truck layer. The batching time can be retrieved from the batching data or offer ticket, for instance. The batching time generally corresponds to a numerical value from which can be retrieved the moment in time at which an initial batch of concrete contained in a drum of a mixer truck is deemed to be ready for delivery. For instance, the batching time can correspond to a date and time entry (e.g., a Coordinated Universal Time entry, a Unix timestamp entry), an elapsed time since readiness of the initial batch, and the like. The batching time can be recorded, stored and/or communicated by the truck layer and/or by the producer layer, depending on the embodiment.

In some embodiments, the coordinator layer can access the initial and/or current temperature associated with a given one of the return concrete loads by retrieving the corresponding temperature(s) on a database made accessible for instance via the producer layer or truck layer. The temperature generally corresponds to a numerical value indicative of the temperature (in Celsius degrees, or equivalently in Fahrenheit degrees) of the return concrete load. The temperature associated with a given one of the return concrete loads can be an initial temperature indicative of the temperature of the initial concrete load as measured at the time of batching, and/or a current temperature indicative of the temperature of the return concrete load obtained via an on-truck measurement. The temperature can be measured once, or preferably measured a plurality of times over time so that the temperature of the return concrete data reflects a current temperature of the return concrete load. It is anticipated that as concrete formation includes an exothermic reaction, heat is generated within the concrete load. Accordingly, it is expected that temperature of the return concrete load will tend to increase over time according to a temperature increase rate, for instance. The temperature increase rate can depend on the concrete recipe associated to each return concrete load. The temperature can be monitored via a plurality of physical measurements within the drum of the mixer truck in some embodiments. In some other embodiments, the current temperature of the return concrete load is calculated by extrapolation/interpolation using the initial temperature, the batching time, the current time and/or the temperature increase rate. The temperature can be recorded, stored and/or communicated by the truck layer and/or by the producer layer, depending on the embodiment.

At step 1604, the coordinator layer accesses regulation data including a maximum temperature for return concrete re-use and a maximum elapsed time since batching. The maximum temperature for concrete re-use generally indicates a maximal temperature limit above which re-use of return concrete load is prohibited in view of some local regulation. Similarly, the maximum elapsed time since batch indicates a maximal elapsed time limit above which re-use of the return concrete load is prohibited in view of some local regulation. In some embodiments, the maximum elapsed time is 180 minutes, preferably 150 minutes and most preferably 120 minutes. In some embodiments, the maximum temperature is 38 degrees Celsius, most preferably 37 degrees Celsius and most preferably 35 degrees Celsius. These numerical values can differ from one jurisdiction to another depending on inside which jurisdictions the mixer trucks are operated, and more specifically on the regulation data associated with the jurisdictions at play.

The regulation data can depend on the local regulation. For instance, when the mixer trucks operate in the United States for instance, the applicable regulations are dictated by the ASTM regulations. Accordingly to the ASTM regulations, a return concrete load should not be used if its temperature is above 38 degrees Celsius (or equivalently 100 degrees Fahrenheit). Moreover, the ASTM regulations dictate that a return concrete load should be reused within a time limit below 90 minutes hours. If the elapsed time since batching is above 90 minutes, but below 8 hours, the return concrete load can be reused provided that a concrete stabilization treatment be applied, which typically, but nor per definition, necessitates the truck mixer to go back to the producer or batch plant for a given period of time. However, in some other jurisdictions, some other regulations data may apply and corresponds regulation data are accessed.

In some embodiments, the coordinator layer can access the regulation data associated with a given one of the mixer trucks by retrieving a location of the mixer truck which then allows the fetching of the applicable regulations. In some embodiments, all the mixer trucks that are monitored are operated within a same jurisdiction hence only one set of regulation data is used. However, in some other jurisdictions, mixer trucks operating in different jurisdictions are monitored thereby requiring different regulation data to be used.

At step 1606, the coordinator layer generates a list of eligible return concrete loads. The list of eligible return concrete loads includes each return concrete load which the return concrete data match said regulation data. For instance, such a match includes either one or both of: i) the temperature of the return concrete load of the return concrete data being below the maximal temperature of the regulation data and ii) the elapsed time of the return concrete data being below the maximum elapsed time of the regulation data.

In some embodiments, the method can include a step of adding a given one of the return concrete loads to the list contingent upon finding a match between the return concrete data of a given return concrete load to the regulation data. In some embodiments, the method can include a step of removing a given one of the eligible return concrete loads off the list contingent upon the return concrete data no longer matching the regulation data. The steps of adding and removing eligible return concrete loads can be integrated in the step of generating the list of eligible return concrete loads. The method can include a step of monitoring an elapsed time since the batching time of the eligible return concrete loads and removing a given one of the eligible return concrete loads contingent upon the elapsed time exceeding the maximum elapsed time since batching. Additionally or alternatively, the method can include a step of monitoring a temperature of the eligible return concrete loads and removing a given one of the eligible return concrete loads contingent upon the temperature exceeding the maximum temperature of the regulation data.

In some embodiments, the step 1602 of accessing the return concrete data and the step 1606 of generating the list of eligible return concrete loads are repeated at a given frequency. As such, if new return concrete loads are rendered accessible to the coordinator layer, the list of eligible return concrete loads can be updated to incorporate newer ones of the return concrete loads. Similarly, if old return concrete loads have been dispatched, the coordinator layer can update the list of eligible return concrete loads to mark or remove the dispatched ones of the return concrete loads off the list.

In some embodiments, the return concrete data further includes an added water content and the regulation data further includes a maximum added water content. In these embodiments, finding a match between the return concrete data of the given return concrete load to the regulation data can include finding a match between the added water content of the return concrete data and the maximum added water content of the regulation data

As shown, the method can have a step 1608 of accessing job tickets including a ticket specification. The job tickets can be received from the buyer layer, for instance. The ticket specification can include a composition requirement, a quantity requirement, a temperature requirement, a batching time requirement and the like depending on the embodiment. In such embodiments, the method has a step 1610 of, upon selecting a given one of the eligible return concrete loads of the list, displaying a sub list of eligible job tickets including at least one of the job tickets which the ticket specification (e.g., one or more of its requirements) is matched by the return concrete data. The sub list of eligible job tickets can offer a quick glance to all the job tickets for which the return concrete load of a given mixer truck could satisfy, for instance. It is intended that the coordinator layer can receive a selection of one or more of the return concrete loads of the list, and upon receiving such a selection remove the selection off the list to avoid a single job ticket to be selected twice by two different mixer trucks. As such, a truck driver or dispatch can skim through regulatory approved return concrete loads and corresponding eligible job tickets at a speed which would not be matched by any paper calculations.

It is envisaged that in some embodiments the list of eligible return concrete loads and/or the sub list of eligible job tickets can be displayed simultaneously or sequentially on a graphical user interface communicatively coupled to the producer layer, the truck layer and/or the buyer layer.

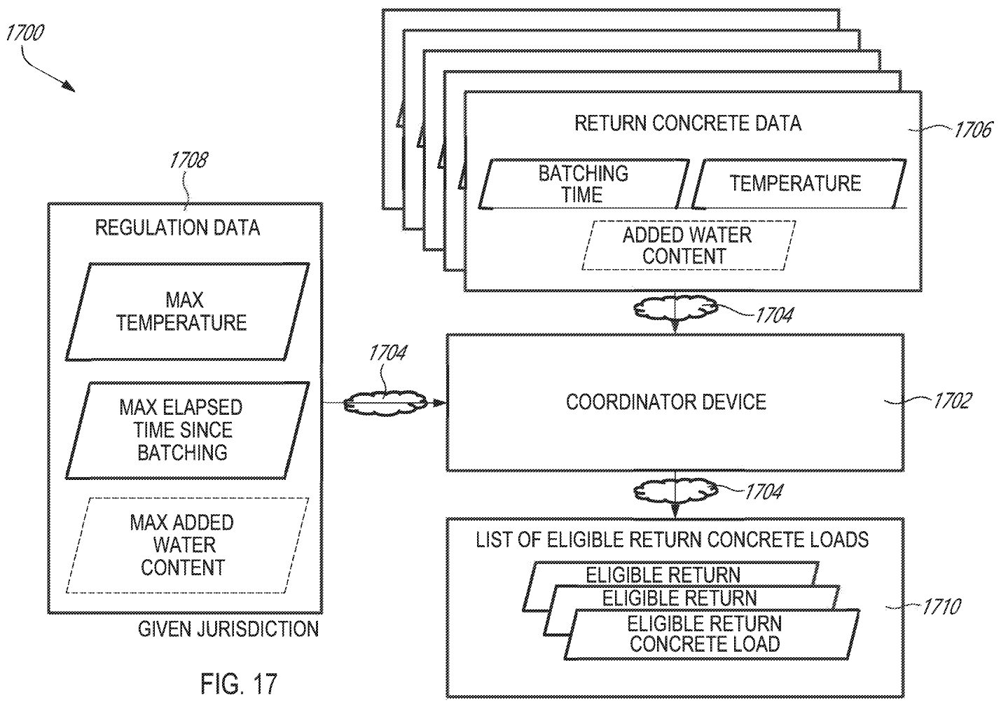

FIG. 17 shows a block diagram of an example of a system 1700 for handling mixer trucks containing return concrete loads. As shown, the system 1700 has a coordinator layer which is communicatively coupled to other connectivity layers such as the buyer layer, the truck layer and the producer layer via one or more internal or external networks such as the Internet. In this example, the coordinator layer can be provided in the form of a coordinator device 1702 communicatively coupled to an external network 1704. The coordinator device 1702 of this example has a processor and a memory having stored thereon instructions that when executed by the processor perform the steps of the method of FIG. 16.

More specifically, the coordinator device 1702 accesses return concrete data 1706 including a batching time, slump, volume and a temperature of the return concrete load and an optional added water content for each one of a plurality of mixer trucks containing return concrete loads. As such, a plurality of such sets of return concrete data are received and processed.

The coordinator device 1702 accesses regulation data 1708 including a maximum temperature for return concrete re-use, a maximum elapsed time since batching and an optional maximum added water content. Although a single set of regulation data is shown in this example, more than one set of regulation data, each associated to a corresponding jurisdiction, can be accessed in some other embodiments.

As shown, the coordinator device 1702 generates a list of eligible return concrete loads 1710. The list of eligible return concrete loads including each return concrete load which the return concrete data match the regulation data. As such, the eligible return concrete loads is generally a sub set of the return concrete loads, as not all return concrete load may match the regulation data.

It is expected that the generated list can be displayed on a graphical user interface made available, for instance, in the cabin of the mixer truck, at an office of the producer or at an office of the buyer. As new return concrete loads are made available to the coordinator device 1702, some new return concrete loads can be added to the list in real time or quasi-real time. When some return concrete loads no longer match the regulation data, some eligible return concrete loads can be removed off the list. For instance, when the temperature exceeds the maximal temperature, a given return concrete load may no longer match the regulation data.

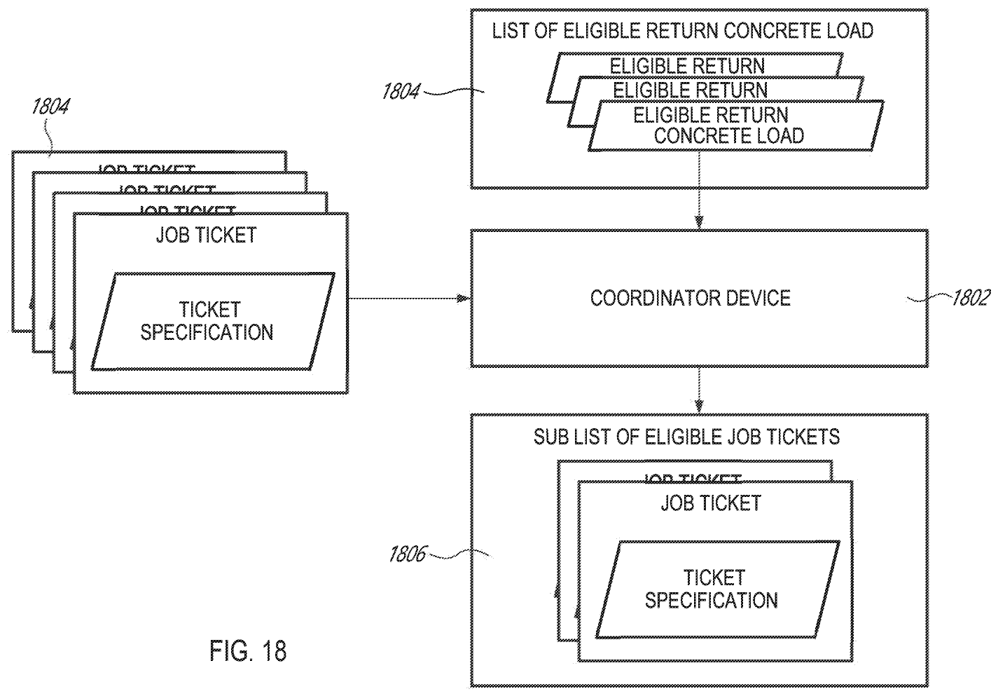

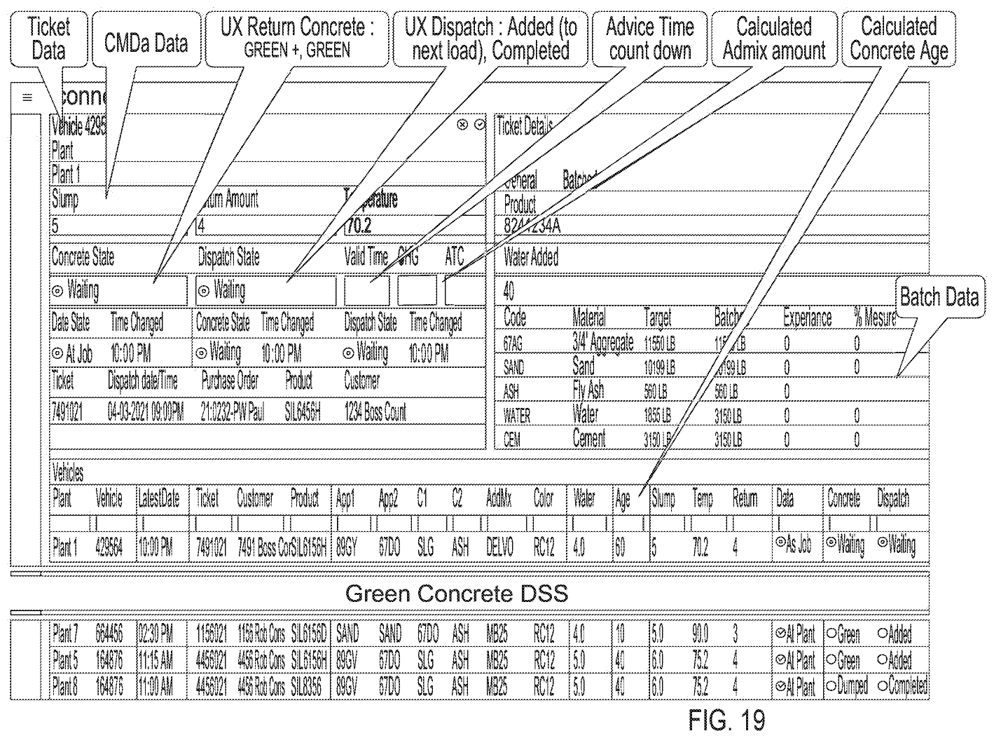

Referring now to FIG. 18, a coordinator device 1802 can also have access to job tickets 1804 each including a ticket specification. The ticket specification can include at least one of a composition requirement and a quantity requirement. The job tickets may be received from the buyer layer in some embodiments. As shown, the coordinator device 1802 may receive a selection 1804 of eligible return concrete loads from the list in which case the coordinator layer may generate a sub list 1806 of eligible job tickets which ticket specification is matched by the return concrete load data. The sub list of eligible job tickets may be displayed on the graphical user interface as well in some embodiments.

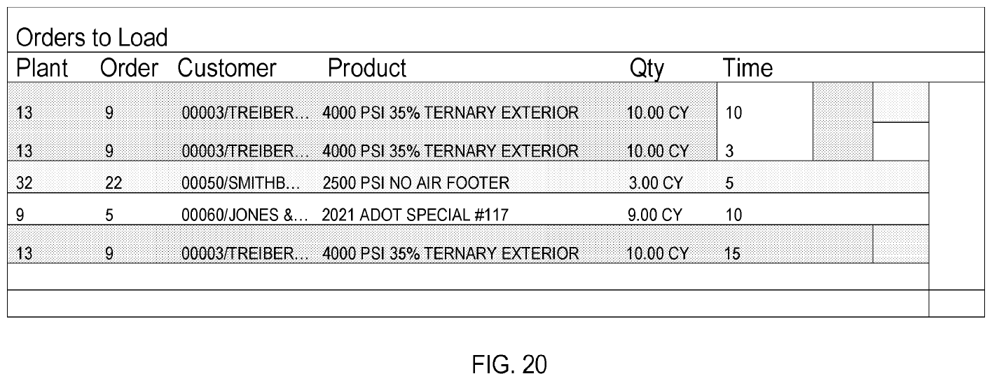

FIG. 19 shows an example of a graphical user interface displaying a list of eligible return concrete loads. As shown, data displayed with respect to each eligible return concrete load includes plant data, mixer truck data, latest date data, age data, water content data, temperature data, composition data and the like. When one of the eligible return concrete loads is selected, as emphasized by the white rectangle selecting the one of the eligible return concrete load, the sub list of eligible job tickets can be prompted, such as shown at inset 19A.

As can be understood, the examples described above and illustrated are intended to be exemplary only. The scope is indicated by the appended claims.

1-40. (canceled)

41. A computer-implemented method for handling mixer trucks containing return concrete loads, the method comprising:
accessing return concrete data including a batching time and a temperature of the return concrete load for each one of a plurality of mixer trucks containing return concrete loads;
accessing regulation data including a maximum temperature for return concrete re-use and a maximum elapsed time since batching; and
generating a list of eligible return concrete loads, the list of eligible return concrete loads including each return concrete load which said return concrete data match said regulation data.

42. The method of claim 41 wherein the maximum elapsed time is at most 180 minutes.

43. The method of claim 41 wherein the maximum temperature is at most 38 degrees Celsius.

44. The method of claim 41 wherein said return concrete data includes an added water content, the regulation data includes a maximum added water content.

45. The method of claim 44 wherein said maximum added water content is at most 6 m³.

46. The method of claim 41 wherein said generating includes adding a given one of the return concrete loads to the list contingent upon said return data matching said regulation data.

47. The method of claim 41 further comprising accessing a plurality of job tickets including a ticket specification and upon selecting a given one of the eligible return concrete loads of the list, displaying a sub list of eligible job tickets including at least one of the job tickets which said ticket specification is matched by the return concrete data.

48. The method of claim 47 wherein said return concrete data includes at least one of composition data indicative of a composition of the return concrete load and quantity data indicative of a quantity of the return concrete load, the ticket specification including at least one of a composition requirement and a quantity requirement.

49. The method of claim 41 wherein said accessing return concrete data and said generating the list of eligible return concrete loads are repeated at a given frequency.

50. The method of claim 41 further comprising displaying the list of eligible return concrete loads on a graphical user interface.

51. A system for handling mixer trucks containing return concrete loads, the system comprising:
a coordinator device communicatively coupled to an external network, the coordinator device having a processor and a memory having stored thereon instructions that when executed by the processor perform the steps of:
accessing return concrete data including a batching time and a temperature of the return concrete load for each one of a plurality of mixer trucks containing return concrete loads;
accessing regulation data including a maximum temperature for return concrete re-use and a maximum elapsed time since batching; and
generating a list of eligible return concrete loads, the list of eligible return concrete loads including each return concrete load which said return concrete data match said regulation data.

52. The system of claim 51 wherein the maximum elapsed time is at most 180 minutes.

53. The system of claim 51 wherein the maximum temperature is at most 38 degrees Celsius.

54. The system of claim 51 wherein said return concrete data includes an added water content, the regulation data includes a maximum added water content.

55. The system of claim 54 wherein said maximum added water content is at most 6 m³.

56. The system of claim 51 wherein said generating includes adding a given one of the return concrete loads to the list contingent upon said return data matching said regulation data.

57. The system of claim 51 further comprising accessing a plurality of job tickets including a ticket specification, and upon selecting a given one of the eligible return concrete loads of the list, displaying a sub list of eligible job tickets including at least one of the job tickets which said ticket specification is matched by the return concrete data.

58. The system of claim 57 wherein said return concrete data includes at least one of composition data indicative of a composition of the return concrete load and quantity data indicative of a quantity of the return concrete load, the ticket specification including at least one of a composition requirement and a quantity requirement.

59. The system of claim 51 wherein said accessing return concrete data and said generating the list of eligible return concrete loads are repeated at a given frequency.

60. The system of claim 51 further comprising displaying the list of eligible return concrete loads on a graphical user interface.
